(19) United States
(12) Patent Application Publication
(10) Pub. No.: US 2025/0276153 A1
(43) Pub. Date: Sep. 4, 2025

(54) EXPANDABLE INTRODUCER APPARATUS AND METHODS OF GUIDING AN OBJECT

(71) Applicant: Medtronic, Inc., Minneapolis, MN (US)

(72) Inventors: Chris MORAN, Mayo (IE); Conleth A. MULLEN, Galway (IE); Marc A. ANDERSON, Barna (IE); Bernard P. MULVIHILL, Mission Viejo, CA (US); Timothy D. FARRELL, Tralee (IE)

(21) Appl. No.: 19/058,512

(22) Filed: Feb. 20, 2025

Related U.S. Application Data
(60) Provisional application No. 63/559,977, filed on Mar. 1, 2024.

(51) Int. Cl. A61M 25/00; A61M 25/02

(52) U.S. Cl. CPC A61M 25/0023; A61M 25/02; A61M 2025/0024; A61M 2025/0035; A61M 2025/0293

(57) ABSTRACT

An expandable introducer apparatus comprising an inner tubular layer comprising an inner lumen and a radially expandable region extending along an elongated axis of the inner lumen and configured to at least partially radially expand relative to the elongated axis from a contracted orientation to an expanded orientation. An outer tubular layer comprising an inner surface bonded to the inner tubular layer, wherein a distal segment of the inner tubular layer extends beyond a distal end of the outer tubular layer. A flexible tubular member comprising a bonded region and a flexible region, wherein an inner surface of the bonded region is bonded to an outer surface of the distal segment of the inner tubular layer, and the flexible region is not bonded to the radially expandable region of the inner tubular layer.

### CROSS-REFERENCE TO RELATED APPLICATIONS

This application claims the benefit of U.S. Provisional Patent Application Ser. No. 63/559,977, filed Mar. 1, 2024, the entire content of which is incorporated herein by reference.

### FIELD

The present disclosure relates generally to an expandable introducer apparatus and methods of guiding an object, and more particularly to expandable introducer apparatus configured to introduce an object into the vasculature of a patient and methods of guiding an object through an expandable introducer apparatus.

### BACKGROUND

During medical procedures, for example, a transcatheter aortic valve replacement, a plurality of objects are commonly introduced into a patient's vasculature. Such objects may include transcatheter valve prosthesis delivery systems. These delivery systems may include various components, for example, a catheter assembly which includes a collapsed valve prosthesis, a guide wire and a plurality of sheaths and tubes. In order to provide percutaneous access for the objects into the patient's vasculature, an introducer is typically employed. Conventional introducers can comprise a hub component, a hemostasis valve, and an elongated tubular member which forms a lumen that runs through a central axis of the elongated tubular member through which the objects enter and exit the patient's vasculature.

During an aortic valve replacement procedure, introducers can accommodate objects of various sizes and shapes while also providing and maintaining support for the objects when they are inserted, removed and/or adjusted. Furthermore, introducers can be reliably percutaneously inserted without damaging the patient's vasculature while reducing the complexity of introducing and removing objects from the vasculature during surgical procedures.

In light of the above, a need exists for an expandable introducer with a flexible tubular member having the capability of expanding and contracting to accommodate objects during medical procedures while maintaining an atraumatic structure to minimize damage to the patient's vasculature.

### SUMMARY

The following presents a simplified summary of the disclosure to provide a basic understanding of some aspects described in the detailed description.

Features of the present disclosure provide an expandable introducer apparatus having a flexible tubular member that seals to an outer surface of an object as it passes therethrough. Providing an introducer apparatus with the flexible tubular member can allow a clinician to reliably insert and remove objects from the vasculature of a patient while reducing injury to the patient's vasculature and reducing or eliminating blood loss.

In aspects, an expandable introducer apparatus comprises an inner tubular layer comprising an inner lumen and a radially expandable region extending along an elongated axis of the inner lumen and configured to at least partially radially expand relative to the elongated axis from a contracted orientation to an expanded orientation. The expandable introducer apparatus further comprises an outer tubular layer comprising an inner surface bonded to the inner tubular layer, wherein a distal segment of the inner tubular layer extends beyond a distal end of the outer tubular layer. The expandable introducer apparatus still further comprises a flexible tubular member comprising a bonded region and a flexible region, wherein an inner surface of the bonded region is bonded to an outer surface of the distal segment of the inner tubular layer. The flexible region is not bonded to the radially expandable region of the inner tubular layer along an axial length of the distal segment. Furthermore, the radially expandable region of the inner tubular layer along the axial length of the distal segment extends under the flexible region of the flexible tubular member.

In further aspects, a method of guiding an object through an inner lumen of an expandable introducer apparatus is provided. The expandable introducer apparatus comprises an inner tubular layer comprising the inner lumen and a radially expandable region extending along an elongated axis of the inner lumen. The expandable introducer apparatus further comprises an outer tubular layer comprising an inner surface bonded to the inner tubular layer, wherein a distal segment of the inner tubular layer extends beyond a distal end of the outer tubular layer. The expandable introducer apparatus further comprises a flexible tubular member comprising a bonded region and a flexible region, wherein an inner surface of the bonded region is bonded to an outer surface of the distal segment of the inner tubular layer. The flexible region is not bonded to the radially expandable region of the inner tubular layer along an axial length of the distal segment. Furthermore, the radially expandable region of the inner tubular layer along the axial length of the distal segment extends under the flexible region of the flexible tubular member. The method comprises distally advancing the object through a proximal end of the inner lumen, wherein a portion of the radially expandable region radially expands from a contracted orientation to an expanded orientation while a corresponding portion of the outer tubular layer expands to accommodate the expanded orientation of the radially expandable region. The method further comprises distally advancing the object past the distal end of the outer tubular layer to radially expand the radially expandable region of the inner tubular layer along the axial length of the distal segment while simultaneously circumferentially stretching the flexible region of the flexible tubular member, wherein the distal segment of the inner tubular layer and the flexible tubular member simultaneously expand to accommodate the object passing through the distal segment of the inner tubular layer. The method further comprises distally advancing the object past a distal end of the inner tubular layer.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 is a schematic view of an exemplary expandable introducer apparatus in accordance with aspects of the present disclosure;

FIG. 2 is a schematic enlarged view of a distal end portion of the expandable introducer apparatus taken at view 2 of FIG. 1 in accordance with aspects of the present disclosure;

FIG. 3 is top schematic view of the distal end portion of the expandable introducer apparatus along line 3-3 of FIG. 2;

FIG. 4 is a schematic cross-sectional view of the distal end portion taken at line 4-4 of FIG. 2;

FIG. 5 is a schematic cross-sectional view of a distal segment of the distal end portion taken at line 5-5 of FIG. 2;

FIG. 6 is a schematic cross-sectional view of another embodiment of a distal segment of the distal end portion taken at line 5-5 of FIG. 2;

FIG. 7 is a schematic cross-sectional view of another embodiment of a distal segment of the distal end portion taken at line 5-5 of FIG. 2;

FIG. 8 is a schematic cross-sectional view of a flexible tip taken at line 8-8 of FIG. 2;

FIGS. 9-12 illustrate example steps in methods of manufacturing the expandable introducer apparatus in accordance with the present disclosure;

FIG. 13 illustrates a schematic perspective view of an exemplary support member that can be provided with the distal segments illustrated in FIGS. 5-7;

FIG. 14 illustrates a schematic side view of the support member of FIG. 13 in a two-dimensional planar state;

FIG. 15 illustrates a schematic perspective view of another embodiment of a support member that can be provided with the distal segments illustrated in FIGS. 5-7;

FIG. 16 illustrates a schematic side view of the support member of FIG. 15 in a two-dimensional planar state;

FIG. 17 illustrates a schematic perspective view of another embodiment of a support member that can be provided with the distal segments illustrated in FIGS. 5-7;

FIG. 18 illustrates a schematic side view of the support member of FIG. 17 in a two-dimensional planar state;

FIG. 19 illustrates a schematic top view of the expandable introducer apparatus with the support member of FIG. 13 circumferentially distributed around an elongated axis of the expandable introducer apparatus;

FIG. 20 illustrated a schematic bottom view of the expandable introducer apparatus with the support member of FIG. 13 circumferentially distributed around the elongated axis of the expandable introducer apparatus;

FIG. 21 illustrates a schematic cross-sectional view generally taken at line 21-21 of FIG. 19 showing a distribution of peaks of the support member of FIG. 13 around the elongated axis of the expandable introducer in accordance with the present discloser;

FIG. 22 illustrates a schematic top view of the expandable introducer apparatus with the support member of FIG. 15 circumferentially distributed around the elongated axis of the expandable introducer apparatus;

FIG. 23 illustrates a schematic bottom of the expandable introducer apparatus with the support member of FIG. 15 circumferentially distributed around the elongated axis of the expandable introducer apparatus;

FIG. 24 illustrates a schematic cross-sectional view generally taken at line 24-24 of FIG. 22 showing a distribution of peaks of the support member of FIG. 15 around the elongated axis of the expandable introducer apparatus in accordance with the present discloser;

FIG. 25 illustrate a schematic top view of the expandable introducer apparatus with the support members of FIGS. 13, 15, and 17 circumferentially distributed around a bottom portion of the elongated axis of the expandable introducer apparatus;

FIG. 26 illustrates a schematic bottom view of the expandable introducer apparatus with the support members of FIGS. 13, 15, and 17 circumferentially distributed around a bottom portion of the elongated axis of the expandable introducer apparatus;

FIG. 27 illustrates a schematic cross-sectional view generally taken at line 27-27 of FIG. 25 showing a distribution of peaks of the support members of FIGS. 13, 15, and 17 around the bottom portion of the elongated axis of the expandable introducer apparatus in accordance with the present discloser;

FIG. 28 illustrates a schematic top view of the expandable introducer apparatus with the support members of FIGS. 13, 15, and 17 biased from radially flaring outward by a constriction of a flexible tubular member;

FIG. 29 illustrates a schematic top view of the expandable introducer apparatus of FIG. 28 with a distal end being radially flared out relative to a proximal end of the support members against a bias of the constriction of the flexible tubular member;

FIG. 30 schematically illustrates an object passing through an inner lumen of an inner tubular layer wherein a portion of a radially expandable region of the inner tubular layer radially expands;

FIG. 31 is a cross-sectional view taken at line 31-31 of FIG. 30, wherein an elongated split of the outer tubular layer is shown to separate to accommodate the expanded orientation of the radially expandable region;

FIG. 32 schematically illustrates the object passing through a distal segment of the inner tubular layer, wherein the distal segment of the inner tubular layer and a flexible tubular member simultaneously expand to accommodate the object passing through the distal segment;

FIG. 33 is a cross-sectional view taken at line 33-33 of FIG. 32;

FIG. 34 schematically illustrates the expandable introducer apparatus in accordance with the present disclosure after the object has passed through a distal end of the expandable introducer; and

FIG. 35 illustrates an exemplary flow chart demonstrating a method of utilizing the expandable introducer apparatus.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art.

Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein—for example up, down, right, left, front, back, top, bottom, upper, lower, etc.—are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” may be used in the following description with reference to one or more valve or stent structures of the prostheses hereof and is intended to convey that the structures are shaped or formed from a material that can be provided with a mechanical memory to return the structure from a compressed or constricted delivery configuration to an expanded deployed configuration or vice versa. Non-exhaustive exemplary self-expanding materials include stainless steel, a pseudo-elastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

In order to facilitate the introduction of the catheter-based delivery system including the heart valve prostheses and various other objects utilized during a procedure, an expandable introducer is first inserted through the skin of the patient. The expandable introducer is typically one which has an expandable region to accommodate the passage of the objects therethrough.

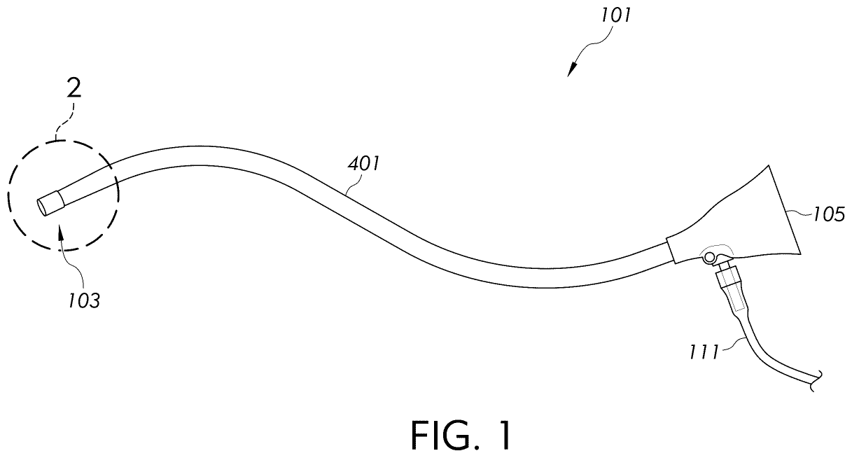

FIG. 1 illustrates an expandable introducer 101 for use with a plurality of objects, such as for example, a catheter-based delivery system. The expandable introducer 101 can be delivered to a desired location within a patient's vasculature for inserting objects therethrough. As shown in FIG. 1, the expandable introducer 101 can comprise an outer tubular layer 401, an inlet 105, and a flush port 111. The object can be inserted through the proximal end of the inlet 105 and then advanced distally along a length of the expandable introducer. The object then exits the expandable introducer 101 through a distal end portion of the expandable introducer 101 where the object is thereafter advanced within the vaculature to the treatment site.

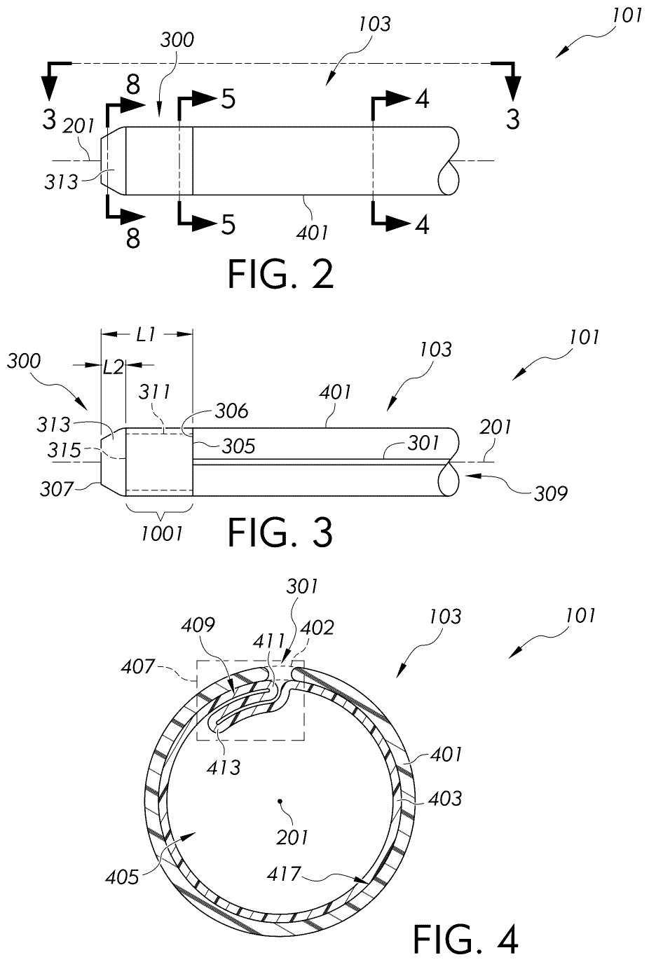

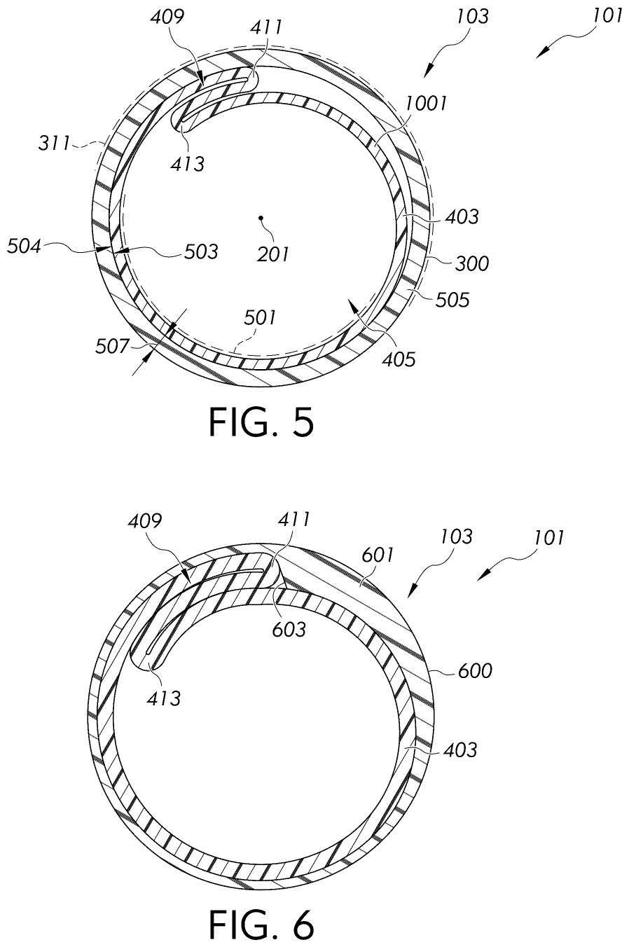

A distal end portion 103 of the expandable introducer 101 is illustrated and discussed with initial reference to FIGS. 2-5. As shown in FIGS. 4-5, an inner tubular layer 403 comprises an inner lumen 405 and a radially expandable region 407 (see FIG. 4) extending along an elongated axis 201 of the inner lumen 405. The radially expandable region 407 may be configured to at least partially radially expand relative to the elongated axis 201 from a contracted orientation (e.g., see FIGS. 4-5) to an expanded orientation (e.g., see FIGS. 31 and 33). The inner tubular layer 403 may be a wide range of materials. For example, the inner tubular layer 403 may be formed from a flexible material, such as for example, tetrafluoroethylene (TFE), Teflon®, polytetrafluoroethylene (PTFE), polyethylene, polyethylene terephthalate (PET), polyester or the like. Further, the inner tubular layer 403 may have a low coefficient of friction to aid in the insertion of objects therethrough.

In some aspects, the radially expandable region 407 of the inner tubular layer 403 can comprise a foldable region 409 that may be configured to at least partially unfold from a contracted orientation to an expanded orientation. In some aspects, the foldable region 409 further comprises an outer fold 411 and an inner fold 413 in the contracted orientation as shown in FIG. 4. The outer fold 411 and the inner fold 413 are configured such that when an object 3101 (e.g., FIG. 14) passes through the inner lumen 405, the outer fold 411 and the inner fold 413 begin to unfold. For example, the outer fold 411 and the inner fold 413 can partially or completely unfold to allow the inner tubular layer 403 to radially expand to accommodate the object 3101 passing through the inner lumen 405.

Throughout the disclosure, the various embodiments of the expandable introducer is illustrated and described being used to introduce the object 3101 schematically illustrated in the drawings. The schematic illustration of the object 3101 is not intended to be limited in size and shape to the illustrated size and shape but may take the size and shape of the object being introduced. Such objects may comprise various types of prosthetic heart valves although the object may comprise other prostheses such as a prosthetic bone fragment, or other prosthetic implant. In further examples, the object may comprise pacemakers or other medical devices or medical implants or other surgical objects to be percutaneously delivered into inserted into the vasculature of the patient.

In some aspects, the expandable introducer 101 can further comprise an outer tubular layer 401 comprising an inner surface 417 bonded to the inner tubular layer 403. In some aspects, as shown in FIGS. 3-4, the outer tubular layer 401 can comprise an optional elongated split 301 extending along the radially expandable region 407 in a direction of the elongated axis 201. As shown in FIG. 4, the optional elongated split 301 can be formed by a first edge facing a second edge. As shown, the first edge can comprise an unobstructed view of the second edge wherein an empty space exists between the first edge and the second edge when the radially expandable region 407 of the inner tubular layer 403 is in the contracted orientation. Although not shown, in some embodiments, the first edge and the second edge of the optional elongated split 301 may abut one another or overlap one another in the contracted orientation in further embodiments. Furthermore, as shown, the optional elongated split 301 can extend along the radially expandable region 407 in the direction of the elongated axis 201 while the radially expandable region 407 of the inner tubular layer 403 is positioned entirely radially inward relative to the optional elongated split 301. Although not shown, in further embodiments, the radially expandable region 407 of the inner tubular layer 403 may partially extend within the optional elongated split 301 wherein the radially expandable region 407 partially or entirely obstructs the view of the first edge from the second edge. Still further, the first edge and the second edge are illustrated as parallel with respect to one another although the first edge (or portions of the first edge) may not be parallel to the second edge (or portions of the second edge) in further embodiments. As further illustrated, the first edge and the second edge are each linear edges that are parallel with one another and each extend in the direction of the elongated axis 201. In further embodiments, although not shown, the first edge and the second edge may comprise curvilinear or other non-linear edge shapes.

The optional elongated split 301 can be configured such that when an object 3101 passes through the inner lumen 405, the optional elongated split 301 circumferentially expands to allow the object 3101 to pass therethrough. As shown by joining segment 402 schematically illustrated in FIG. 4, any of the embodiments of the disclosure may provide the outer tubular layer 401 without the previously-described elongated split. Rather, as schematically represented by the joining segment 402, the outer tubular layer 401 may comprise a cross section taken perpendicular to the elongated axis 201 that is continuous around the entire periphery without any breaks that would otherwise by caused by an elongated split. Furthermore, such continuity can extend along a substantial portion of the length, such as the entire length, of the outer tubular layer 401 in a direction of the elongated axis 201. Furthermore, while the joining segment 402 is illustrated as having a thickness that is substantially the same as the thickness as the remainder of the periphery of the outer tubular layer, different thicknesses may be provided in further embodiments. For instance, the thickness of the joining segment 402 can be thinner at the radially expandable region 407 to allow easier expansion of the joining segment 402 at the foldable region 409 during expansion of the inner tubular layer 403.

The outer tubular layer 401 may be a wide range of materials. For example, the outer tubular layer 401 may be formed from a shape memory material or a resilient material, such as for example, polyurethane (e.g., Pellethane®, Elasthane™, Texin®, or Tecothane®), Pebax®, polyethylene or the like. Additionally, and/or alternatively, the outer tubular layer 401 may further include material(s) that allow the outer tubular layer 401 to be radiopaque to facilitate the detection of the expandable introducer 101 by electromagnetic radiation.

Referring to FIGS. 3 and 5, in some aspects, a distal segment 1001 of the inner tubular layer 403 extends beyond a distal end 306 of the outer tubular layer 401. In some aspects, the expandable introducer 101 further comprises a flexible tubular member 300 that comprises a proximal end 305 that can abut, and in some examples engage, the distal end 306 of the outer tubular layer 401 although the proximal end 305 of the flexible tubular member 300 may not abut the distal end 306 of the outer tubular layer 401 (e.g., may be spaced apart) in further embodiments. The flexible tubular member 300 further comprises a distal end 307, and a length L1 extending from the proximal end 305 to the distal end 307 in a direction of the elongated axis 201. For example, the length L1 can be from about 5 mm to about 7 mm. In aspects, the flexible tubular member 300 can comprise a flexible tip 313 that distally extends beyond a distal end 315 of the inner tubular layer 403. The flexible tip 313 can comprise a length L2 extending from the distal end 315 of the inner tubular layer 403 to the distal end 307 of the flexible tubular member 300 in the direction of the elongated axis 201. For example, the length L2 of the flexible tip 313 can be from about 0.5 mm to about 3 mm, for example, from about 1 mm to about 1.5 mm.

The flexible tubular member 300 may be made from a variety of suitable materials. For example, the flexible tubular member 300 can be made from a highly flexible elastic material having a high columnar strength. High columnar strength refers to the ability of the material to withstand buckling or collapsing. In this way, the material should permit the flexible tubular member 300 and the flexible tip 313 to expand and contract, enabling them to accommodate the object 3101 passing therethrough while being capable of preventing the flexible tip 313 from collapsing. For example, a high columnar strength will prevent the flexible tip 313 from collapsing inward towards the proximal end 305 of the flexible tubular member 300 when the object 3101 is proximally retracted relative to the flexible tubular member 300 when removing the object 3101 from the patient's vasculature and from the expandable introducer 101. The material can be a wide range of materials. For example, the flexible tubular member 300 may be made from Thermoplastic Polyurethane (TPU), Pellethane, Texin, and/or any other similarly suitable materials.

As shown in FIG. 5, in some aspects the flexible tubular member 300 comprises a bonded region 501 and a flexible region 311 (schematically represented by dashed lines). The bonded region 501 and flexible region 311 are each schematically represented by dashed lines in FIG. 5 that are intended to represent an example of the circumferential extent of the bonded and flexible regions at an arcuate interface between the flexible tubular member 300 and the distal segment 1001 of the inner tubular layer 403. For example, bonded region 501 is understood to comprise an inner surface 503 of the flexible tubular member 300 bonded to an outer surface 504 of the distal segment 1001 of the inner tubular layer 403 along the circumferential portion of the arcuate interface between the inner surface 503 and the outer surface 504 that is parallel to the illustrated arcuate broken line indicating the bonded region 501 of the arcuate interface. Furthermore, in some embodiments, the bonded region can extend a length (e.g., the entire length) of the distal segment 1001 of the inner tubular layer 403 in the direction of the elongated axis 201.

As further shown in FIG. 5, flexible region 311 is understood to comprise the inner surface 503 of the flexible tubular member 300 that is not bonded to the outer surface 504 of the distal segment 1001 of the inner tubular layer 403 along the circumferential portion of the arcuate interface between the inner surface 503 and the outer surface 504 that is parallel to the illustrated arcuate broken line indicating the flexible region 311 of the arcuate interface. As such, within the flexible region, the inner surface 503 of the flexible tubular member 300 is not bonded to the radially expandable region 407 of the inner tubular layer 403. Furthermore, as shown in FIG. 3, the flexible region 311, in some embodiments, can extend a length (e.g., the entire length) of the distal segment 1001 of the inner tubular layer 403 in the direction of the elongated axis 201.

In aspects, the radially expandable region 407 of the distal segment 1001 of the inner tubular layer 403 extends under the flexible region 311 of the flexible tubular member 300. For example, the radially expandable region 407 can extend under the flexible region 311 of the flexible tubular member 300 along the entire length of the distal segment 1001 in the direction of the elongated axis 201. In this way, when the object 3101 passes through the inner lumen 405, the flexible region 311 and the radially expandable region 407 will be able to simultaneously radially expand together to accommodate the object 3101 passing therethrough.

In the embodiment illustrated in FIG. 5, the flexible tubular member 300 surrounds the inner tubular layer 403 with an inner cross-sectional surface profile (see inner surface 503) matching an outer cross-sectional surface profile (see outer surface 504) of the distal segment 1001 of the inner tubular layer 403. In this way, the flexible tubular member 300 can be bonded to the inner tubular layer 403 within the bonded region 501 as discussed above. In some aspects, the flexible tubular member 300 comprises a circular cross-sectional area 505 with a uniform wall thickness 507 circumscribing the distal segment 1001 of the inner tubular layer 403.

In some aspects, the bonded region 501 can be formed utilizing a number of suitable bonding processes. For example, the bonding process may include but is not limited to a reflow bonding process or a laser bonding process, such as for example, by pressing the flexible tubular member 300 to the outer cross-sectional surface profile of the distal segment 1001 of the inner tubular layer 403 and applying a heat source to form the bonded region 501. In this way, the bonded region 501 is formed where the flexible tubular member 300 becomes joined to the outer cross-sectional surface profile of the distal segment 1001 where the heat source is applied. The bonding procedure is not limited to a reflow or laser bonding process. For example, the bonding procedure may comprise an adhesive bonding process or a process that involves manufacturing the flexible tubular member 300 as a monolithic structure of the expandable introducer 101.

FIG. 6 illustrates additional embodiments, of a flexible tubular member 600 comprising an inner circumferential flared segment 601 comprising a flared end 603 abutting a folded end of the outer fold 411 of the foldable region 409. In some embodiments, the inner circumferential flared segment 601 may extend (e.g., continuously extend) the entire length “L1” of the flexible tubular member 600 (see “L1” in FIG. 3). Alternatively, the inner circumferential flared segment 601 may extend less than the entire length of the flexible tubular member 600. For example, the inner circumferential flared segment 601 may extend along the distal segment 1001 of the inner tubular layer 403 without extending into the length L2 of the flexible tip 313.

As further shown in FIG. 6, the flared end 603 can comprise a thickness that gradually circumferentially increases (e.g., in the illustrated counterclockwise direction) toward the outer fold 411. Further, the outer fold 411 circumferentially extends toward the flared end 603 of the flared segment (e.g., in the illustrated clockwise direction) such that the folded end of the outer fold 411 faces the flared end 603. As shown, in some embodiments, the folded end of the outer fold 411 can abut the flared end 603 wherein the flared end 603 can provide a seat for the folded end of the outer fold 411. The inner circumferential flared segment 601 can fill a negative space that may otherwise be formed by the outer fold 411. In this way, as shown in FIG. 6, the inner circumferential flared segment 601 may provide the outer circumferential surface of the flexible tubular member 600 with a more uniform profile and can support the flexible tubular member 600 to resist radial deformation and stress concentrations that may otherwise impact the profile of the flexible tubular member 600. The circumferential flared segment 601 can therefore provide a more uniform or circumferential expansion of the flexible tubular member 600.

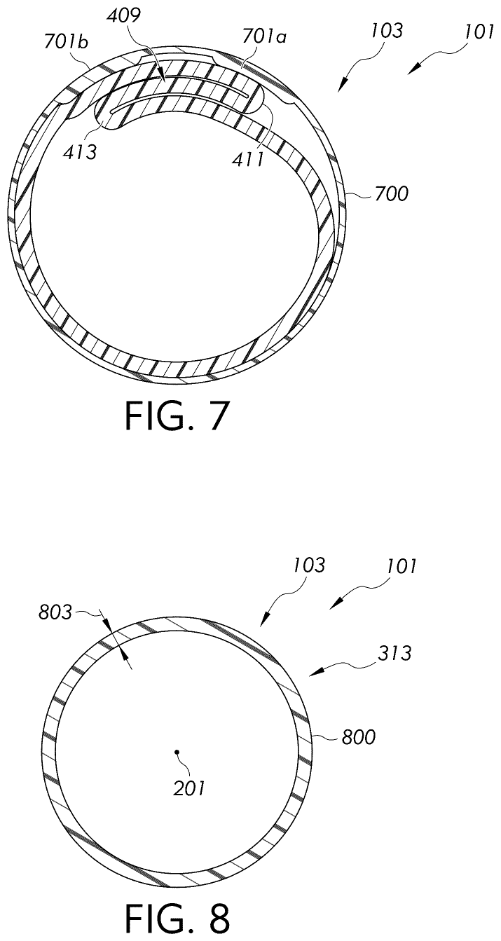

FIG. 7 illustrates an additional embodiment of a flexible tubular member 700 comprising a first inner protrusion 701a aligned with the folded end of the outer fold 411 of the foldable region 409 and a second inner protrusion 701b aligned with the folded end of the inner fold 413 of the foldable region 409. In some embodiments, the first and second inner protrusion 701a, 701b may extend (e.g., continuously extend) the entire length “L1” of the flexible tubular member 700 (see “L1” in FIG. 3). Alternatively, the first and second inner protrusion 701a, 701b may extend less than the entire length of the flexible tubular member 700. For example, the first and second inner protrusion 701a, 701b may extend along the distal segment 1001 of the inner tubular layer 403 without extending into the length L2 of the flexible tip 313.

As further illustrated in FIG. 7, the first and second inner protrusions 701a, 701b can comprise thickened areas of the flexible tubular member 700 that are spaced apart from one another by an intermediate area of reduced thickness relative to the first and second inner protrusions 701a, 701b. Furthermore, as shown, in some embodiments, a majority of the circumference of the flexible tubular member 700 can extend with a reduced thickness relative to the first and second inner protrusions 701a, 701b that circumferentially extends from the first inner protrusion 701a to the second inner protrusion 701b. As shown, the majority of the circumference of the flexible tubular member 700 and the intermediate area extending between the first and second inner protrusions 701a, 701b can comprise substantially the same reduced thickness although different thicknesses may be provided in further embodiments. As shown, the first inner protrusion 701a can be aligned to press against the folded end of the outer fold 411 while the second inner protrusion 701b can be aligned to press against the folded end of the inner fold 413. In this way, a more uniform circumferential expansion of the flexible tubular member 700 can be maintained subsequent to the bonding process.

The flexible tubular member 300, 600, 700 can be manufactured utilizing any number of manufacturing methods. For example, the flexible tubular member 300, 600, 700 can be created utilizing an extrusion process, and/or a molding process. In some embodiments, the flexible tubular member 300, 600, 700 can be formed by radio-frequency molding, or radio-frequency forming of an extrusion. In this way, the flexible tubular member 300, 600, 700 can be manufactured to conform to a variety of material characteristics and design specifications, such as for example, a material with a high columnar strength and low modulus of elasticity to facilitate the expansion and contraction of the flexible region 311 while avoiding failure (e.g., collapsing).

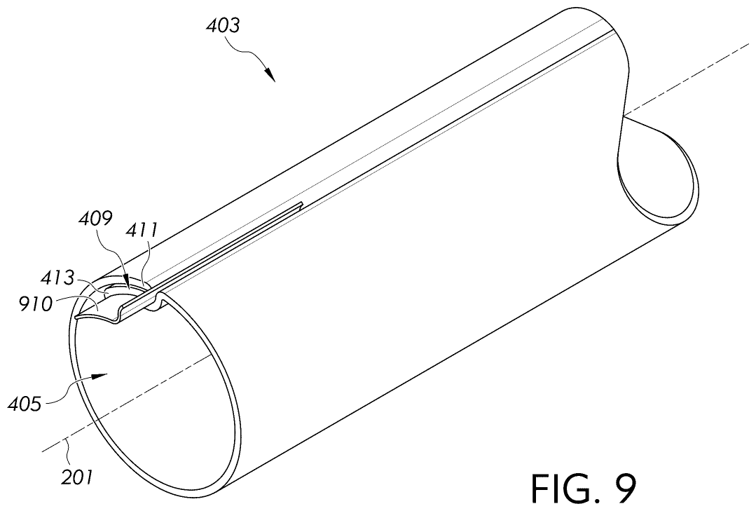

As shown in the cross-section of FIG. 8, in some embodiments, the flexible tip 313 can comprise a circular cross-sectional area 800 with a uniform wall thickness 803 although nonuniform wall thicknesses may be provided in further embodiments. In other aspects, the flexible tip 313 may comprise any other shape(s) required to accommodate the object 3101 passing through the distal end 315 of the inner tubular layer 403.

FIGS. 9-12 illustrate exemplary methods of manufacturing the expandable introducer 101 in accordance with the present disclosure. FIG. 9 illustrates a perspective view of the inner tubular layer 403 comprising the foldable region 409 comprising the outer fold 411 and the inner fold 413. Further depicted is the inner lumen 405 extending along the elongated axis 201. In aspects, the foldable region 409 can extend along the elongated axis 201 of the inner lumen 405 (e.g., along the entire length of the inner tubular layer 403 in the direction of the elongated axis 201). In some aspects, a gap can be formed between the outer fold 411 and the inner fold 413 and extends along the elongated axis 201.

As explained previously, during a reflow or laser bonding process, heat may be applied to surfaces of materials to be bonded. In this way, the layers of materials begin to melt as a result of the directed heat source. Accordingly, the surfaces of the materials begin to contact one another. Once the layers of the melted materials solidify, the layers form a strong bond with one another.

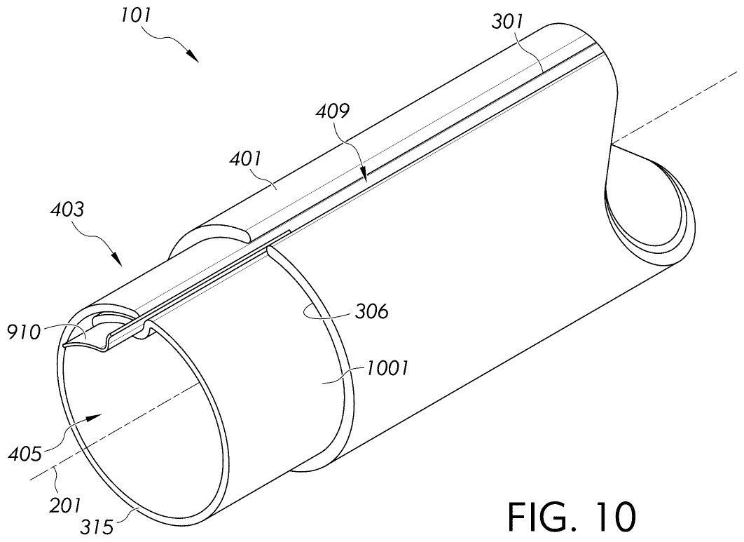

In aspects, in order to prevent the bonding of the foldable region 409, for example, portions of the flexible tubular member 300 from melting and flowing into the inner fold 413 and/or portions of the inner fold 413 and the outer fold 411 melting together, an inner shim 910 can be placed within the gap between the outer fold 411 and the inner fold 413. For example, the inner shim 910 can be slidably positioned through the gap between the outer fold 411 and the inner fold 413 and extend into the inner fold 413 from outside of the inner tubular layer 403. The inner shim 910 can extend along the elongated axis 201 of the inner lumen 405 any length required to prevent a bonding of the inner fold 413 together and/or a bonding of the outer fold 411 to the inner fold 413. For example, the inner shim 910 can extend a total length of the inner tubular layer 403. In an alternative embodiment, the inner shim 910 can extend half the length of the inner tubular layer 403. In some examples, as shown in FIG. 10, the inner shim 910 can have a length that is greater than the length of the distal segment 1001 in the direction of the elongated axis 201. For example, as shown, in some embodiments, the proximal end of the inner shim 910 can extend proximally past the distal end 306 of the outer tubular layer 401 while also extending distally past the distal end 315 of the inner tubular layer 403.

FIG. 10 illustrates a perspective view of the expandable introducer 101 comprising the outer tubular layer 401 positioned over the inner tubular layer 403 with the optional elongated split 301 of the outer tubular layer 401 aligned with the foldable region 409 of the inner tubular layer 403. Although not illustrated in FIG. 10, in further embodiments, the outer tubular layer 401 may comprise a circular cylindrical tube without an elongated split. Indeed, the outer tubular layer 401 may continuously extend as schematically represented by the joining segment 402 illustrated in FIGS. 4 and 30-31. Furthermore, as discussed above, the joining segment 402 may have a reduced thickness and can be aligned with the foldable region 409 of the inner tubular layer 403 to facilitate expansion of the joining segment 402 at the location where the inner tubular layer 403 unfolds during expansion of the inner tubular layer 403. As further illustrated, the outer tubular layer 401 can be positioned relative to the inner tubular layer such that the distal segment 1001 of the inner tubular layer 403 extends beyond the distal end 306 of the outer tubular layer 401.

In some aspects, the flexible tubular member 300, 600, 700, shown in FIGS. 2-3, and 5-8, can be bonded to the distal segment 1001. The distal segment 1001 can be any number of suitable lengths. For example, the distal segment 1001 can be a length of 6 mm. In this way, the flexible tubular member 300, 600, 700 will have a 6 mm bonding length. In this non-limiting example, the proximal end 305 of the flexible tubular member 300 can abut the distal end 306 of the outer tubular layer 401 and extend distally in the distal direction along the elongated axis 201 of the inner lumen 405 as described in FIGS. 2-3, and 5.

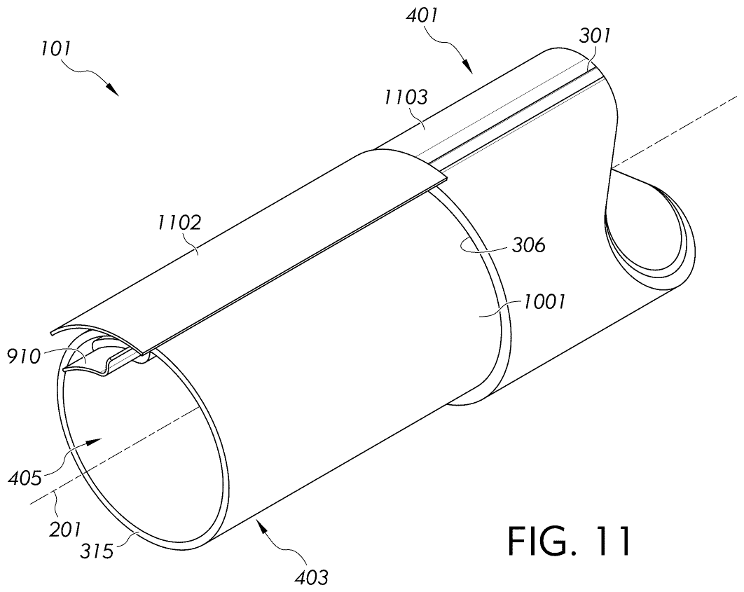

FIG. 11 illustrates a perspective side view of the expandable introducer 101 with an outer shim 1102 comprising a proximal portion positioned on a distal portion of an outer surface 1103 of the outer tubular layer 401 and extending along the elongated axis 201 of the inner lumen 405 and over an outer area of the distal segment 1001. For example, as shown, in some embodiments, the outer shim 1102 can extend over the outer area along the entire length of the distal segment 1001. Due to the positioning of the outer shim 1102, the flexible tubular member 300 will not bond to the distal segment 1001 of the inner tubular layer 403 at the outer area covered by the outer shim 1102 and the flexible tubular member will not bond the facing split edges of the outer tubular layer 401 together. In this way, the flexible region 311 will be created to allow the foldable region 409 to freely unfold and facing edges to spread apart so that the inner tubular layer 403 can achieve the expanded orientation. In some aspects, the bonded region 501 may be calculated to bond to half the circumference of the distal segment 1001 to the flexible tubular member 300. For example, the circumferential extent of the bonded region 501 can be calculated by determining the circumference of the distal segment 1001 and dividing the determined circumference number in half. In other aspects, the outer shim 1102 can be positioned to cover the folded end of the outer fold 411 and the folded end of the inner fold 413. In other aspects, the outer shim 1102 can be any other suitable width required to form the flexible region 311.

In some aspects, the outer shim 1102 can be a variety of suitable lengths. For example, in aspects, the outer shim 1102 can correspond to the length of the distal segment 1001, such as for example, in some embodiments the distal segment 1001 can be 6 mm from the distal end 306 of the outer tubular layer 401 and the outer shim 1102 can also be 6 mm and extend distally from the distal end 306 of the outer tubular layer 401. In other non-limiting examples, the outer shim 1102 can be any other length necessary to form the flexible region 311. In the illustrated embodiment, the outer shim 1102 can be longer than the length of the distal segment 1001 to facilitate removal of the outer shim 1102 after bonding the flexible tubular member to the distal segment 1001. For example, as mentioned previously, a proximal portion of the outer shim 1102 can be positioned on the distal portion of the outer surface 1103 of the outer tubular layer 401. At the same time, in some embodiments, the outer shim can extend along the elongated axis 201 of the inner lumen 405 while extending over the outer area of the distal segment, and distally past the distal end 315 of the inner tubular layer 403. The outer shim 1102 can therefore prevent undesired bonding at the foldable region 409 and the distal portion of the elongated split 301, if provided. As such, once formed, the foldable region 409 is free to unfold while the facing edges of the elongated split 301, if provided, are free to spread apart so that the inner tubular layer 403 can achieve the expanded orientation.

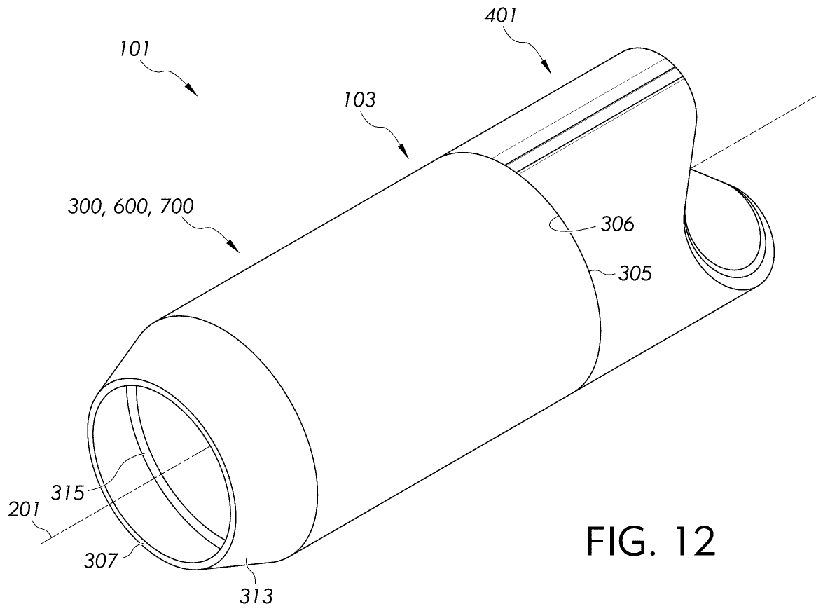

FIG. 12 illustrates a perspective view of the expandable introducer 101 shown in FIG. 11 with the flexible tubular member 300, 600, 700 bonded to the distal segment 1001 of the inner tubular layer 403. As shown, the flexible tip 313 of the flexible tubular member 300, 600, 700 extends beyond the distal end 315 of the inner tubular layer 403. The inner shim 910 and outer shim 1102 have been removed since the bonding process has been completed. For example, after bonding the flexible tubular member 300, 600, 700 to the distal segment 1001, the inner shim 910 may be gently pulled out from between the gap of the outer fold 411 and the inner fold 413, utilizing extra precaution to avoid tearing or ripping the outer fold 411 or the inner fold 413. The inner shim 910 and the outer shim 1102 may be any number of suitable materials that prevent the previously-described bonding. For example, the inner shim 910 and/or the outer shim 1102 can be made from a thin sheet of ceramic, metal, polyimide, PTFE and/or the like.

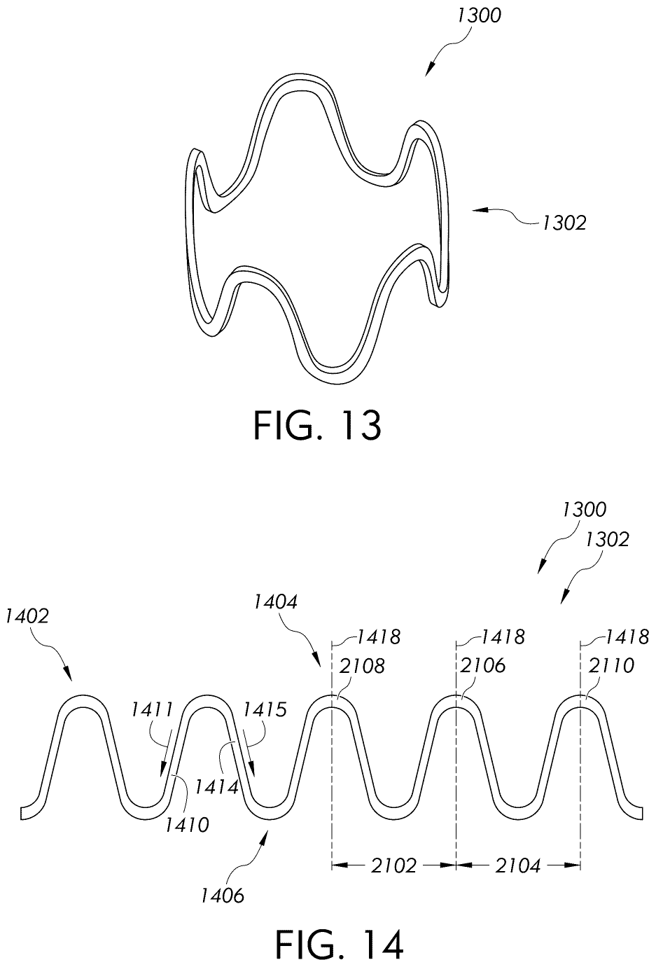

In some aspects, FIGS. 13, 15, and 17 show perspective views of example support members 1300, 1500, 1700 that can be provided with the distal segment 1001 to increase the columnar strength of the inner tubular layer 403, the flexible tubular member 300, 600, 700, and/or the outer tubular layer 401. Indeed, providing the inner tubular layer 403 with the support member 1300, 1500, 1700 can inhibit, such as prevent, the flexible tubular member 300, 600, 700, the inner tubular layer 403, and/or the outer tubular layer 401 from collapsing inwardly (e.g., buckling), such as when an object 3101 is proximally moved back through the distal end 315 and into the inner lumen 405 to be proximally removed from the proximal end of the expandable introducer 101. In some examples, it may be especially beneficial to provide the distal segment 1001 with the support member 1300, 1500, 1700 when larger objects 3101 (e.g., objects with a diameter that is larger than the inner diameter of the inner lumen 405 in the contracted orientation) are expected to be inserted through the expandable introducer 101.

As shown in FIGS. 13, 15, and 17, the support member 1300, 1500, 1700 can comprise a wire structure 1302, 1502, 1702. In some embodiments, the wire structure can comprise an elongated wire that is extruded or drawn to form the elongated wire that extends along an elongated axis of the elongated wire. The wire structure can comprise any number of suitable cross-sectional areas along a cross-section taken perpendicular to the elongated axis of the elongated wire. For example, suitable cross-sectional areas can comprise a circular cross-sectional area, a square cross-sectional area, an octagonal cross-sectional area, or other polygonal or curvilinear cross-sectional areas.

As further shown in FIGS. 13, 15, and 17, the wire structure 1302, 1502, 1702 can be arranged in a tubular configuration. As shown, the support member 1300, 1500, 1700 can comprise a circular cylindrical tubular configuration although a frustoconical cylindrical tubular configuration can be provided in further embodiments. Still further, as shown, the wire structure 1302, 1502, 1702 can be provided with a series of peaks and valleys arranged in a circumferential sinusoidal pattern.

For illustration purposes, FIGS. 14, 16, and 18 illustrate the wire structure 1302, 1502, 1702 of FIGS. 13, 15, and 17 in a two-dimensional planar state. As shown, in some aspects, the wire structure 1302, 1502, 1702 can comprise a continuous wire structure although the wire structure can comprise segments that are joined together in further embodiments. In some embodiments, a wire can be continuously bent or otherwise formed into one of the 2-dimensional shapes illustrated in FIGS. 14, 16, and 18, and then the bent wire structure can be wound around a cylindrical mandrel into the tubular shape illustrated in FIGS. 13, 15, and 17. The two free ends of the wire structure can then be integrally attached together (e.g., by welding) to form the support member 1300, 1500, 1700. In another embodiment, the wire can be bent around a cylindrical mandrel to form the peaks and valleys while also forming the tubular shape illustrated in FIGS. 13, 15 and 17. The two free ends of the wire structure can then be integrally attached together (e.g., by welding) to form the support member 1300, 1500, 1700. In still further embodiments, the support members 1300, 1500, 1700 can be fabricated by 3-dimensional printing, casting, machining, or other forming techniques to obtain to form the support member 1300, 1500, 1700.

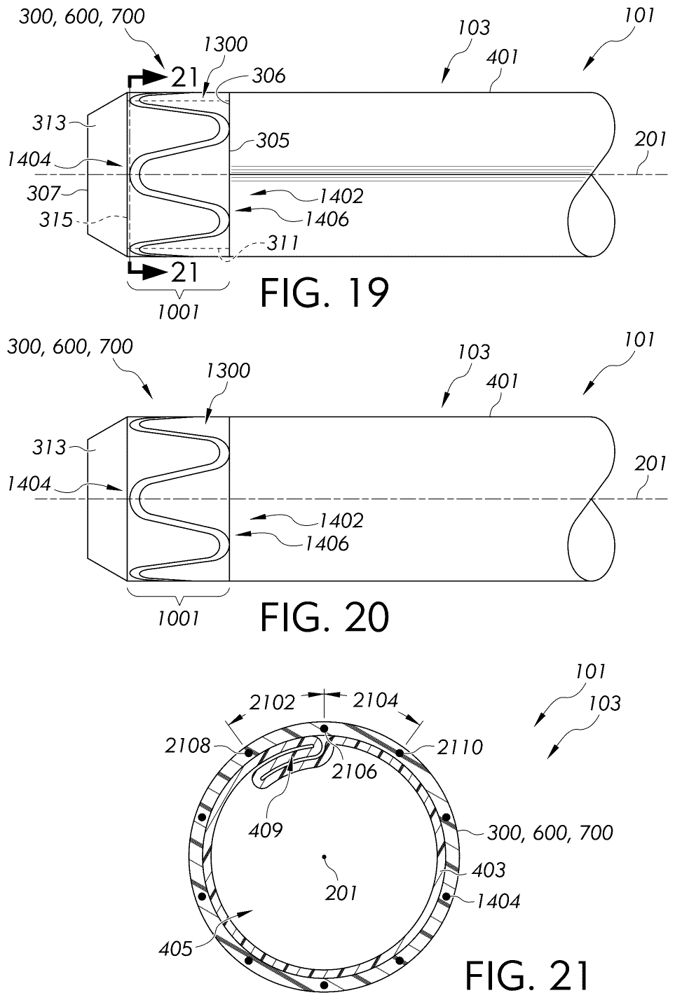

With additional reference to FIGS. 19-21, in some aspects, the plurality of peaks 1402 of the wire structure 1302 can be circumferentially distributed around the elongated axis 201. For example, the support member 1300 can be circumferentially distributed around the entire elongated axis 201 (e.g., distributed 360 degrees around the elongated axis 201). For example, as shown in FIG. 19, the support member 1300 is shown circumscribing the elongated axis 201 from a top view of the expandable introducer 101 (e.g., with a view of the flexible region 311). FIG. 20 shows the support member 1300 circumscribing the elongated axis 201 from a bottom view of the expandable introducer 101. In this way, the support member 1300 will provide support to both the flexible region 311 and the bonded region 501 (see FIG. 5). In further aspects, the plurality of peaks 1402 can further comprise a plurality of distal peaks 1404 and a plurality of proximal peaks 1406.

In some aspects, each peak of the plurality of distal peaks 1404 can converge in a distal direction (see FIGS. 19 and 20). For example, an apex of each peak of the plurality of distal peaks 1404 can be pointing towards the distal direction (e.g., in a direction towards the distal end 307 of the flexible tubular member 300, 600, 700). In some aspects, each peak of the plurality of proximal peaks 1406 can converge in a proximal direction (see FIGS. 19 and 20). For example, an apex of each peak of the plurality of proximal peaks 1406 can be pointing towards the proximal direction (e.g., in a direction towards the distal end 306 of the outer tubular layer 401). In some examples, each distal peak of the plurality of distal peaks 1404 can be laterally positioned between a corresponding adjacent pair of proximal peaks of the plurality of proximal peaks 1406.

In some aspects, the plurality of distal peaks 1404 and the plurality of proximal peaks 1406 can comprise V-shaped peaks. In some non-limiting examples, with reference to FIG. 14, any distal peak of the plurality of distal peaks 1404 can comprise a first wire segment 1410 extending from the distal peak in a first direction 1411 to connect to a corresponding first adjacent proximal peak and a second wire segment 1414 extending from the distal peak in a second direction 1415 to connect to a corresponding second adjacent proximal peak. In further non-limiting examples, the above described plurality of proximal peaks 1406 and distal peaks 1404 can be arranged to form the wire structure 1302 in a sinusoidal pattern of alternating distal and proximal peaks circumferentially arranged to circumscribe the elongated axis 201.

In some aspects, the plurality of distal peaks 1404 and the plurality of proximal peaks 1406 can comprise rounded peaks, pointed peaks, flat peaks and/or any other suitable peaks. In some examples, one or more distal peaks of the plurality of distal peaks 1404 can comprise different distal peaks than that of the other distal peaks. For example, one distal peak of the plurality of distal peaks can be a V-shaped distal peak (e.g., a pointed peak), while another different distal peak of the plurality of distal peaks 1404 can be a flat distal peak. Any suitable combination of distal peaks (e.g., V-shaped peaks, rounded peaks, pointed peaks, flat peaks) may be utilized. In some examples, one or more proximal peaks of the plurality of proximal peaks 1406 can comprise different proximal peaks than that of the other proximal peaks. For example, one proximal peak of the plurality of proximal peaks 1406 can be a V-shaped proximal peak (e.g., a pointed peak), while another different proximal peak of the plurality of proximal peaks 1406 can be a flat proximal peak. Any suitable combination of proximal peaks for the plurality of proximal peaks 1406 (e.g., V-shaped peaks, rounded peaks, pointed peaks, flat peaks) may be utilized. In some aspects, the plurality of distal peaks 1404 and the plurality of proximal peaks 1406 can comprise the same combination of peaks, such as those described above (e.g., V-shaped peaks, rounded peaks, pointed peaks, flat peaks). In other aspects, the plurality of distal peaks 1404 and the plurality of proximal peaks 1406 can comprise a different combination of peaks (e.g., V-shaped peaks, rounded peaks, pointed peaks, flat peaks). For example, the plurality of distal peaks 1404 can comprise V-shaped peaks while the plurality of proximal peaks 1406 can comprise flat peaks, or any other suitable combination, such as those described above.

As shown in FIGS. 19 and 20, the support member 1300 can extend along the axial length of the distal segment 1001 of the inner tubular layer 403 in a direction of the elongated axis 201. For example, the support member 1300 can extend along the elongated axis 201 from the distal end 315 of the inner tubular layer 403 to the distal end 306 of the outer tubular layer 401. In other examples, the support member 1300 can extend along only a portion of the axial length of the distal segment 1001 of the inner tubular layer 403. For example, the support member 1300 can extend from the distal end 315 of the inner tubular layer 403 to half the axial length of the distal segment 1001. In other examples, the support member can extend any suitable distance along the axial length of the distal segment 1001 of the inner tubular layer 403 (e.g., more than half the axial length or less than half the axial length).

In some aspects, as best shown in FIG. 21, a circumferential distance between each adjacent pair of peaks of the plurality of peaks 1402 can be substantially equal in an unexpanded orientation. For example, as shown in FIG. 21, a first circumferential distance 2102 between a first distal peak 2106 (represented generally by a dot) and an adjacent second distal peak 2108 can be substantially equal to a second circumferential distance 2104 between the first distal peak 2106 and an adjacent third distal peak 2110 in an unexpanded orientation. In some examples, all adjacent circumferential distances between adjacent distal peaks can be substantially equal in an unexpanded orientation as shown in FIG. 21. Likewise, all circumferential distances between adjacent proximal peaks can be substantially equal in an unexpanded orientation. Furthermore, in some embodiments, all adjacent circumferential distances between adjacent distal peaks and adjacent proximal peaks can be substantially equal in an unexpanded orientation as will be appreciated by the embodiment illustrated in FIGS. 19-21.

FIG. 14 further illustrates the distance between each adjacent pair of peaks of the plurality of peaks 1402 being substantially equal. For example, FIG. 14 shows dotted lines 1418 bisecting the first distal peak 2106, the second distal peak 2108, and the third distal peak 2110 of the plurality of distal peaks 1404. The first circumferential distance 2102 and the second circumferential distance 2104 are shown respectively from the first distal peak 2106 to the second distal peak 2108 and from the first distal peak 2106 to the third distal peak 2110. While FIG. 14 shows the wire structure 1302 in a two-dimensional planar state, the circumferential distance between adjacent pairs of peaks will also be equal when the wire structure 1302 is in a tubular shape as previously described.

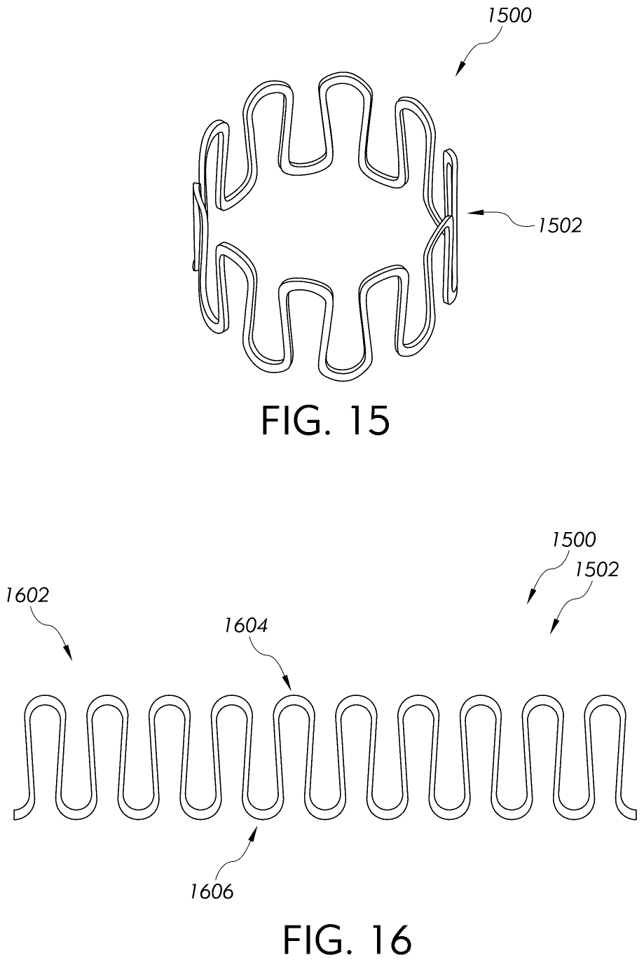

FIGS. 15-16 illustrate a support member 1500 that can have features, unless otherwise stated, that are similar or identical to features of the support member 1300 illustrated and described with reference to FIGS. 13-14. As shown in FIGS. 15 and 16, in some aspects, the wire structure 1502 of the support member 1500 can comprise a plurality of peaks 1602 arranged in a sinusoidal pattern but with a smaller pitch (e.g., the distance from one peak to an adjacent peak) than the sinusoidal pattern of peaks 1402 of the wire structure 1302 of the support member 1300. In this way, the plurality of distal peaks 1604 and/or the plurality of proximal peaks 1606 can comprise a greater number of peaks per unit length (e.g., total length, half length, and/or the like) of the wire structure 1502 than that of the plurality of distal peaks 1404 and/or the plurality of proximal peaks 1406 per unit length (e.g., total length, half length, and/or the like) of the wire structure 1302. Increasing the number of peaks per unit length may be useful to increase the rigidity of the flexible tubular member 300, 600, 700, the inner tubular layer 403, and/or the outer tubular layer 401. In this way, the flexible tubular member 300, 600, 700, the inner tubular layer 403, and/or the outer tubular layer 401 will have a greater resistance to buckling, such as for example, while removing an object 3101 from the expandable introducer 101.

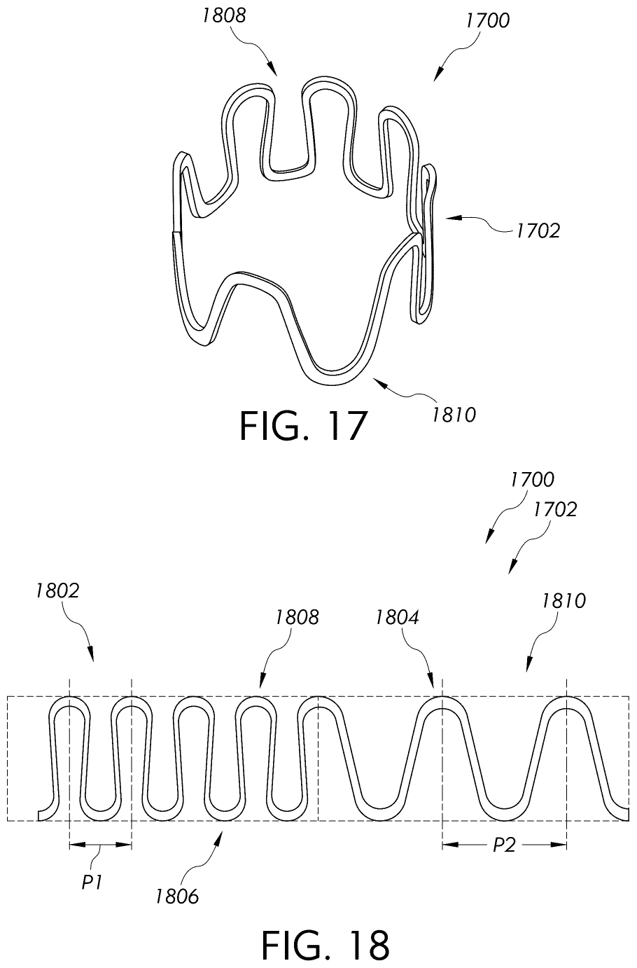

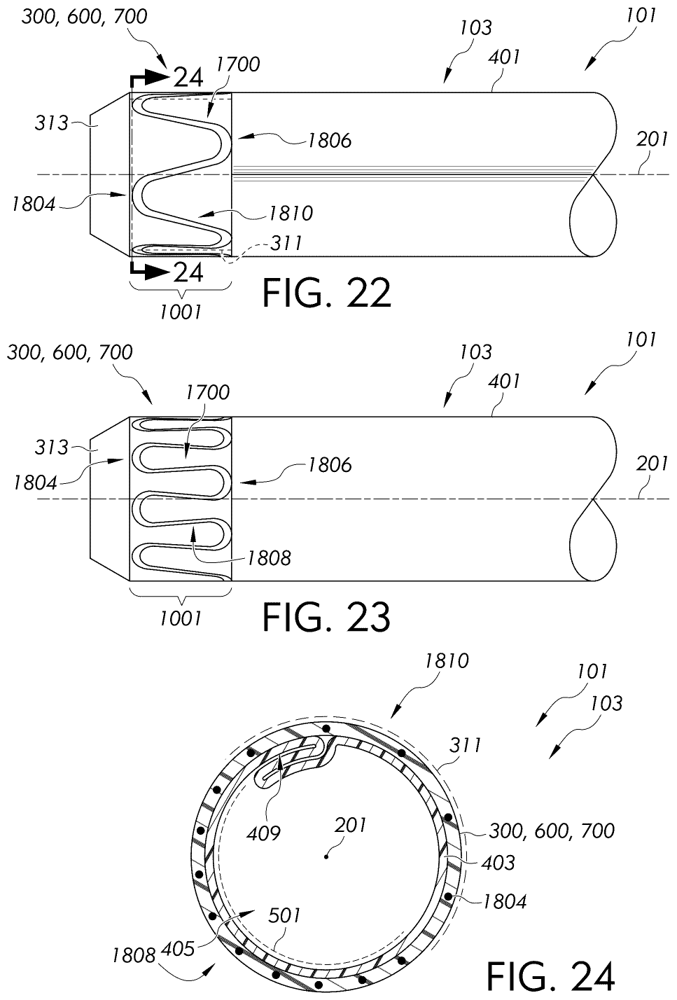

FIGS. 17 and 18 illustrate another embodiment of a support member 1700 that can have features, unless otherwise stated, that are similar or identical to features of one or both of the support members 1300, 1500 illustrated and described with reference to FIGS. 13-16. For example, the support member 1700 can comprise a wire structure 1702 comprising a plurality of peaks 1802 arranged in a sinusoidal pattern. In some similar aspects to the support members 1300, 1500, the plurality of peaks 1802 can be circumferentially distributed around an elongated axis 201 (see FIGS. 22-24). In further aspects, the plurality of peaks 1802 can further comprise a plurality of distal peaks 1804 and a plurality of proximal peaks 1806.

However, as shown in FIGS. 17 and 18, in some aspects a first portion 1808 (shown generally by the broken-line box in FIG. 18) of the wire structure 1702 can comprise a smaller pitch (e.g., a distance between peaks) than a second portion 1810 (shown generally by the broken-line box in FIG. 18) of the wire structure 1702. In some examples, as shown in FIG. 18, a pitch P1 between two adjacent distal peaks 1804 of the first portion 1808 can be lesser than a pitch P2 between two different adjacent distal peaks 1804 of the second portion 1810. As further shown in FIG. 18, in some embodiments, the pitch between two adjacent proximal peaks 1806 of the first portion 1808 can be the same as the pitch P1 between two adjacent distal peaks 1804 of the first portion 1808. As further illustrated, in some embodiments, the pitch between two adjacent proximal peaks 1806 of the second portion 1810 can be the same as the pitch P2 between two different adjacent distal peaks 1804 of the second portion 1810.

In some aspects, a number of peaks of the plurality of peaks 1802 (e.g., distal peaks 1804 and/or proximal peaks 1806) in the bonded region 501 can be greater than a number of peaks (e.g., distal peaks 1804 and/or proximal peaks 1806) of the plurality of peaks 1802 in the flexible region 311. For example, as shown in FIG. 24, the flexible region 311 can comprise less distal peaks 1804 per unit of circumferential length than the bonded region 501. Accordingly, more or less peaks can be provided in accordance with this embodiment described above. Furthermore, it should be understood that while FIG. 24 shows a cross-sectional area of FIG. 22 along a portion of the expandable introducer 101 where the plurality of distal peaks 1804 would be, and thus the description describes the aforementioned principles in terms of distal peaks, the same may be true for the plurality of proximal peaks 1806 (e.g., having less peaks in the flexible region than the bonded region).

As further shown in FIGS. 22 and 23, the portion of the support member 1700 that comprises a greater pitch (e.g., pitch P2 of the second portion 1810) can be positioned around the elongated axis 201 such that the portion of the support member 1700 with less peaks (e.g., the second portion 1810) can be circumferentially adjacent to the flexible region 311 (see FIG. 22). In contrast, in some aspects, the portion of the support member 1700 that comprises a smaller pitch (e.g., pitch P1 of the first portion 1808) can be positioned around the elongated axis 201 such that the portion of the support member 1700 with more peaks (e.g., the first portion 1808) can be circumferentially adjacent to the bonded region 501 (see FIG. 22). This may be especially beneficial to provide the flexible tubular member 300, 600, 700, the inner tubular layer 403, and/or the outer tubular layer 401 with greater rigidity (e.g., to resist buckling when removing an object 3101 from the expandable introducer 101) in the bonded region where circumferential expansion is not a concern. Furthermore, an increased pitch (decreased number of peaks per unit circumferential length) can be provided in the flexible region to provide reduced circumferential stiffness in the flexible region 311 to avoid an undesired resistance to circumferential expansion in in the flexible region 311 while still providing a sufficient level of rigidity to avoid buckling.

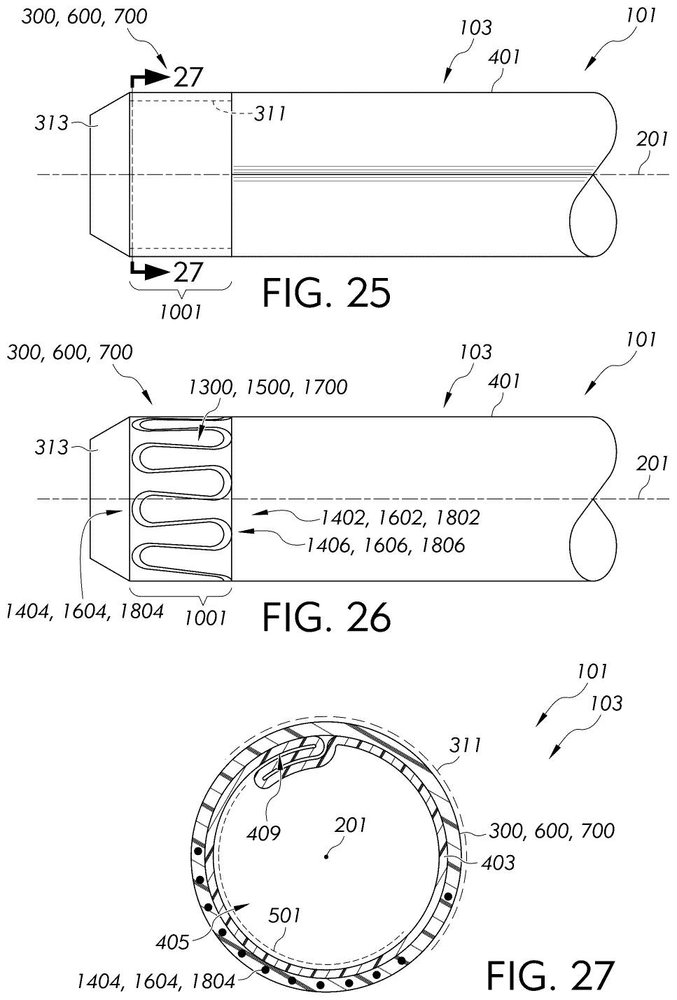

Turning to FIGS. 25-27, as shown, in some aspects, the support member 1300, 1500, 1700 can be limited to only circumferentially extending along a portion (e.g., the bonded region 501) of the flexible tubular member 300, 600, 700. More specifically, FIG. 26 shows the support member 1300, 1500, 1700 circumferentially extending only along the bottom of the expandable introducer 101 (e.g., along a portion of the bonded region 501). As illustrated in FIG. 25, the support member 1300, 1500, 1700 does not circumferentially extend into the flexible region 311. In this way, as shown in FIG. 27, the plurality of peaks 1402, 1602, 1802 will only be circumferentially distributed around the elongated axis 201 circumferentially adjacent to the bonded region 501. It should be understood that while the support member 1300, 1500, 1700 is described above as being tubular, the support member 1300, 1500, 1700 can be adapted to only circumferentially extent along the bonded region 501 in a number of suitable ways. For example, a length (e.g., relative to the two-dimensional planar state shown in FIGS. 14, 16, and 18) or circumference (e.g., relative to the tubular state shown in FIGS. 13, 15, and 17) of the support member 1300, 1500, 1700 can be reduced such that it only extends along the bonded region 501. In this way, the support member 1300, 1500, 1700 will be an arcuate shape extending along the bonded region 501. The embodiment of FIGS. 25-27 can provide maximum stiffness in the bonded region that may be sufficient to resist buckling while avoiding any resistance to expansion of the flexible region by the support member 1300, 1500, 1700 to facilitate passage of the object through the inner lumen.

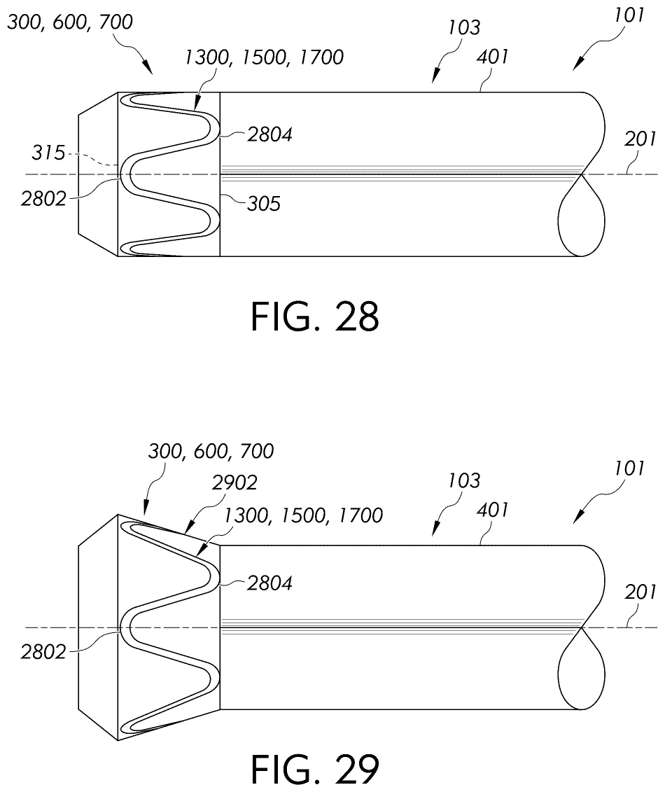

As shown in FIGS. 28 and 29, in aspects, the support member 1300, 1500, 1700 can be biased from radially flaring outward by a constriction of the flexible tubular member 300, 600, 700. For example, in some aspects, the support member 1300, 1500, 1700 can be configured to self-expand from a constricted state to its natural, expanded state. As shown in FIG. 28, the flexible tubular member 300, 600, 700 can constrict the support member 1300, 1500, 1700 to its contracted state and therefore bias the support member 1300, 1500, 1700 against radially flaring outward. When the flexible tubular member 300, 600, 700 expands (e.g., when an object 3101 enters the flexible tubular member 300, 600, 700) the support member 1300, 1500, 1700 will tend to self-expand towards its natural, expanded state, as shown in FIG. 29. Thus, support member 1300, 1500, 1700 can be provided, as described above, to increase the rigidity to inhibit, such as prevent, buckling while the tendency of the support member 1300, 1500, 1700 to self expand from the contracted state can help reduce the force necessary for the object to expand the flexible region.

In some aspects, as shown in FIG. 29, the distal end 2802 of the support member 1300, 1500, 1700 can comprise a greater diameter than the proximal end 2804 of the support member 1300, 1500, 1700 when in its natural, expanded state. In some aspects, when the support member 1300, 1500, 1700 is in the constricted state (e.g., as a result of the flexible tubular member 300, 600, 700 constricting the support member 1300, 1500, 1700), the proximal end 2804 and the distal end 2802 can comprise the same or similar diameters. In some aspects, when the support member 1300, 1500, 1700 is expanded towards its natural, expanded state (e.g., when an object 3101 enters the flexible tubular member 300, 600, 700), the flexible tubular member 300, 600, 700 can comprise a distally flared frustoconical surface 2902. In aspects, the distally flared frustoconical surface 2902 extend from the proximal end 305 of the flexible tubular member 300, 600, 700 to the distal end 315 of the inner tubular layer 403. In this way, for example, the flexible tubular member 300, 600, 700 can gradually increase in diameter from the proximal end 305 of the flexible tubular member 300, 600, 700 to the distal end 315 of the inner tubular layer 403 along the distally flared frustoconical surface 2902.

In some aspects, when the support member 1300, 1500, 1700 comprises an arcuate shape (as described above with reference to FIGS. 25-27) to extend only along the bonded region 501, the proximal end 2804 and the distal end 2802 can comprise same or similar arc lengths when in the constricted state. In some such examples, the distal end 2802 will radially flare out relative to the proximal end 2804 when the support member 1300, 1500, 1700 is expanded towards its natural, expanded state.

Furthermore, in some such examples, the distally flared frustoconical surface 2902 can circumferentially extends around only a portion of the flexible tubular member 300, 600, 700 (e.g., less than 360 degrees around the flexible tubular member 300, 600, 700). For example, the distally flared frustoconical surface 2902 can circumferentially extend only around the bonded region 501 of the flexible tubular member 300, 600, 700.

In some aspects, the support member 1300, 1500, 1700 can be heat set to create a self-expanding material. In some aspects, the distal end 2802 and the proximal end 2804 of the support member 1300, 1500, 1700 can be heat set to impart the above described radial flare. For example, by heat setting the material of the support member 1300, 1500, 1700 in a conical shape, the support member 1300, 1500, 1700 will be biased to return to its conical shape. In this way, the support member 1300, 1500, 1700 will exhibit a propensity to naturally revert from the constricted state to its natural, expanded state.

In some aspects, by making the support member 1300, 1500, 1700 a self-expanding material, it can help to reduce the amount of force required to stretch the flexible region 311 of the flexible tubular member 300, 600, 700 (e.g., when an object 3101 enters the flexible tubular member 300, 600, 700). For example, because the support member 1300, 1500, 1700 will have a tendency to want to radially expand but is prohibited from doing so by the constriction of the flexible tubular member 300, 600, 700, the support member 1300, 1500, 1700 can decrease the amount of force required for an object 3101 to stretch the flexible region 311 of flexible tubular member 300, 600, 700 by providing an additional source of radial expansion. Furthermore, as the distal end is flared outwardly, in the natural, expanded state, even less force is necessary at the distal end when proximally retracting the object back through the catheter at the end of the procedure, thereby minimizing the potential of buckling at the point of entry.

In some aspects, the support member 1300, 1500, 1700 can comprise nitinol. In some aspects, the nitinol can provide a self-expanding support member 1300, 1500, 1700. This is not meant to be limiting, any other suitable material may be utilized for the support member 1300, 1500, 1700.

In some aspects, the support member 1300, 1500, 1700 can be embedded within the flexible tubular member 300, 600, 700. For example, the flexible tubular member 300, 600, 700 can be formed with the support member 1300, 1500, 1700 contained therein, such as for example, by injection molding the flexible tubular member 300, 600, 700 around the support member 1300, 1500, 1700. Any other suitable method known in the art may be utilized to embed the support member 1300, 1500, 1700 within the flexible tubular member 300, 600, 700. In some aspects, the support member 1300, 1500, 1700 can be embedded within the inner tubular layer 403. For example, the support member 1300, 1500, 1700 can be embedded within the distal segment 1001 of the inner tubular layer 403. In other aspects, the support member 1300, 1500, 1700 can be provided as an insert and circumferentially positioned within the inner lumen 405, such as for example, circumferentially positioned within the inner lumen 405 and extending along the distal segment 1001 (e.g., from distal end 315 of the inner tubular layer 403 to the proximal end 305 of the flexible tubular member). In some such examples, the insert can be circumferentially positioned between the flexible tubular member and the distal segment 1001. For example, the support member 1300, 1500, 1700 can be circumferentially positioned over top of the distal segment 1001 of the inner tubular layer 403 (e.g., not embedded within the distal segment 1001) and running underneath the flexible tubular member 300, 600, 700. In some such examples, the flexible tubular member 300, 600, 700 can be bonded (e.g., such as described previously) to the distal segment 1001 of the inner tubular layer 403, after circumferentially positioning the support member 1300, 1500, 1700 around the distal segment 1001. In other aspects, the support member 1300, 1500, 1700 can be circumferentially positioned over top and around the flexible tubular member 300, 600, 700. In yet another aspect, the support member 1300, 1500, 1700 can be embedded within a portion of the flexible tubular member 300, 600, 700, such as for example, a circumferential upper portion and/or a lower portion of the flexible tubular member 300, 600, 700 (e.g., such as to only be distributed around the bonded region 501 as described with reference to FIGS. 25-27).

In some aspects, the support member 1300, 1500, 1700 can axially extend past the proximal end 305 of the flexible tubular member 300, 600, 700. For example, the support member 1300, 1500, 1700 can axially extend into and/or over a portion of the outer tubular layer 401 (e.g., can be embedded within a portion of the outer tubular layer 401 and/or contact a portion of the outer tubular layer 401). In further such examples, the support member 1300, 1500, 1700 can be positioned within the inner lumen 405 and/or embedded into the inner tubular layer 403, as described above, and axially extend past the proximal end 305 of the flexible tubular member 300, 600, 700 and into the outer tubular layer 401. In yet another such example, the support member 1300, 1500, 1700 can be embedded within or positioned overtop of the flexible tubular member 300, 600, 700 and can axially extend past the proximal end 305 of the flexible tubular member 300, 600, 700 (e.g., can be embedded within a portion of the outer tubular layer 401 and/or contact a portion of the outer tubular layer 401).

It should be understood that the example pitches of the peaks illustrated and described with reference to FIGS. 13-29 are merely exemplary. Accordingly support members 1300, 1500, 1700 described herein may comprise any range of pitches, such as for example, peaks with a greater or reduced pitch and/or various pitch patterns. Furthermore, it should be realized that the support member 1300, 1500, 1700 can comprise a greater or lesser number of peaks than that shown in FIGS. 13-29.

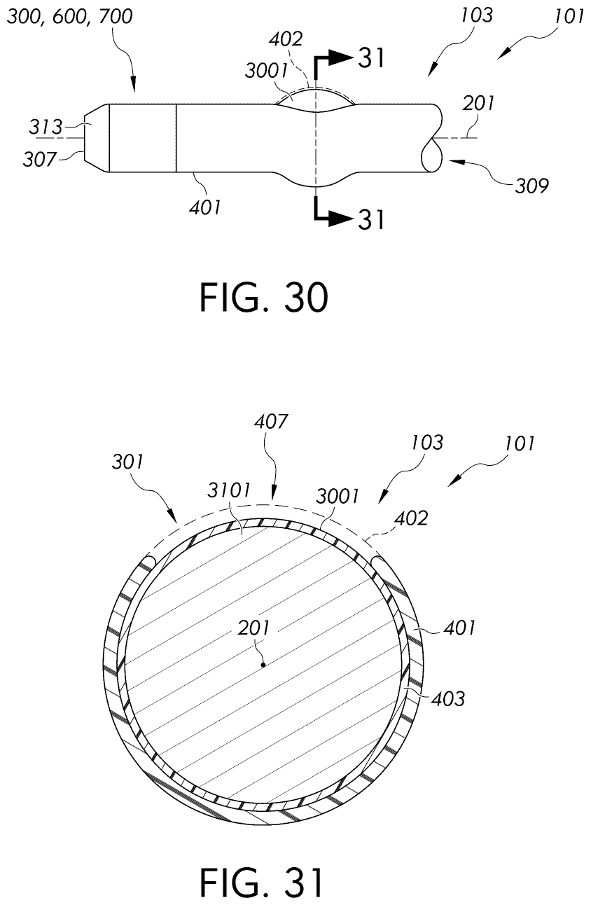

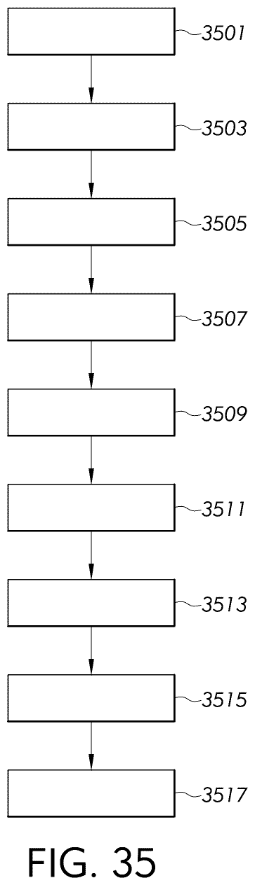

FIGS. 30-35 will now describe a method of guiding the object 3101 through the expandable introducer 101 comprising the flexible tubular member 300, 600, 700 with the understanding that similar or identical methods may be provided in the other embodiments of the disclosure. More specifically, FIG. 35 illustrates an exemplary flow chart showing example steps for guiding the object 3101 through the expandable introducer 101.

Referring to FIG. 35, during step 3501, an incision is made at an anatomical location of the patient's vessel. For example, a transfemoral approach that involves making an incision in the groin and operates as a passageway for guiding a catheter-based delivery system to a target site (e.g., a diseased or defective heart valve).

As shown during step 3503, the method can further comprise percutaneously introducing a guide wire into the patient's vasculature via the incision made during step 3501. The guide wire can then be carefully guided through the patient's vasculature to the target site. For example, the guide wire can be guided through the incision in the groin of the patient to the location of the diseased or defective heart valve (e.g., aortic valve). The guide wire can serve as a path for subsequent objects to travel along when inserted into the patient via the incision made during step 3501.

During step 3505, the method can comprise percutaneously inserting the expandable introducer 101 into the incision made during step 3501 and sliding the expandable introducer 101 into the patient's vasculature. For example, the expandable introducer 101 can slide along the guide wire introduced during step 3503. In some aspects, the expandable introducer 101 can have a dilator introduced within the inner lumen 405 to facilitate the expansion of a passageway within the patient's vessel. As shown, in some embodiments, the flexible tip 313 can comprise a constricted conical shape. In some aspects, the constricted conical shape of the flexible tip 313 axially covers the distal end 315 of the inner tubular layer 403 and thereby provides a beneficial atraumatic feature that helps avoid injury or irritation to the patient's vasculature when inserting the expandable introducer 101.

Next, during step 3507 the method can comprise distally advancing the object 3101 through the proximal end of the inlet 105 into the proximal end 309 of the inner lumen 405. As shown in FIGS. 30-31, a portion 3001 of the radially expandable region 407 radially expands from a contracted orientation to an expanded orientation while a corresponding portion of the outer tubular layer 401 expands to accommodate the expanded orientation or the radially expandable region of the inner tubular layer 403. In embodiments with an elongated split 301, the expansion of the corresponding portion of the outer tubular layer 401 can comprise separating the elongated split 301, as shown in FIGS. 30-31, to accommodate the expanded orientation of the inner tubular layer 403. The object 3101 is schematically represented in cross-section in FIGS. 31 and 33. Alternatively, if the outer tubular layer 401 is not provided with an elongated split, a portion of the outer tubular layer 401 (e.g., the joining segment 402) or the entire circumference of the outer tubular layer 401 may expand to accommodate the expanded orientation of the inner tubular layer 403.

As shown in FIG. 31, in embodiments with an elongated split 301, the radially expandable region 407 of the inner tubular layer 403 can radially expand while the facing edges of the elongated split 301 of the outer tubular layer 401 circumferentially spread apart such that the inner tubular layer 403 achieves the expanded orientation to accommodate the object 3101. Indeed, as shown, the facing edges of the elongated split 301 can spread apart from one another such that the width of the split increases to accommodate the expansion of the inner tubular layer 403. The portion 3001 appears bulged as a result of the object 3101 passing through the inner lumen 405 and thus conforms to the shape of the object 3101 passing therethrough. Alternatively, if the outer tubular layer 401 is provided as a continuous tube (e.g., with the joining segment 402) without the elongated split, portions of the outer tubular layer 401 or the entire outer circumference of the outer tubular layer 401 can stretch to accommodate the expansion of the inner tubular layer 403. For example, the circumferential width of the joining segment 402 can increase from the width shown in FIG. 4 to the expanded width shown in FIG. 31 to accommodate expansion of the inner tubular layer 403. In further embodiments, the entire circumferential width of the outer tubular layer 401 (e.g., not just a portion of the outer tubular layer in the vicinity of the foldable region 409) can stretch to accommodate the expansion of the inner tubular layer 403. As illustrated in FIG. 31, the foldable region 409 has fully unfolded. For example, the outer fold 411 and the inner fold 413 have fully unfolded such that the inner lumen 405 has fully radially expanded to its maximum diameter. In some other aspects, the outer fold 411 and the inner fold 413 can partially radially expand (e.g., wherein the inner lumen 405 only partially radially expands). The foldable region 409 can expand to any range of suitable diameters. For example, the foldable region 409 can have an outer fold 411 and an inner fold 413 configured to accommodate the largest diameter object 3101 expected to pass through the inner lumen 405. For example, a fold length between the outer fold 411 and the inner fold 413 can be increased (e.g., by forming a larger outer fold 411 and/or inner fold 413). In this way, the maximum expanded outer diameter of the inner tubular layer 403 will be increased, thereby allowing larger objects to pass through the inner lumen 405. As further illustrated, the elongated split 301 has separated (or the joining segment 402 has expanded) to accommodate the expansion of the radially expandable region.

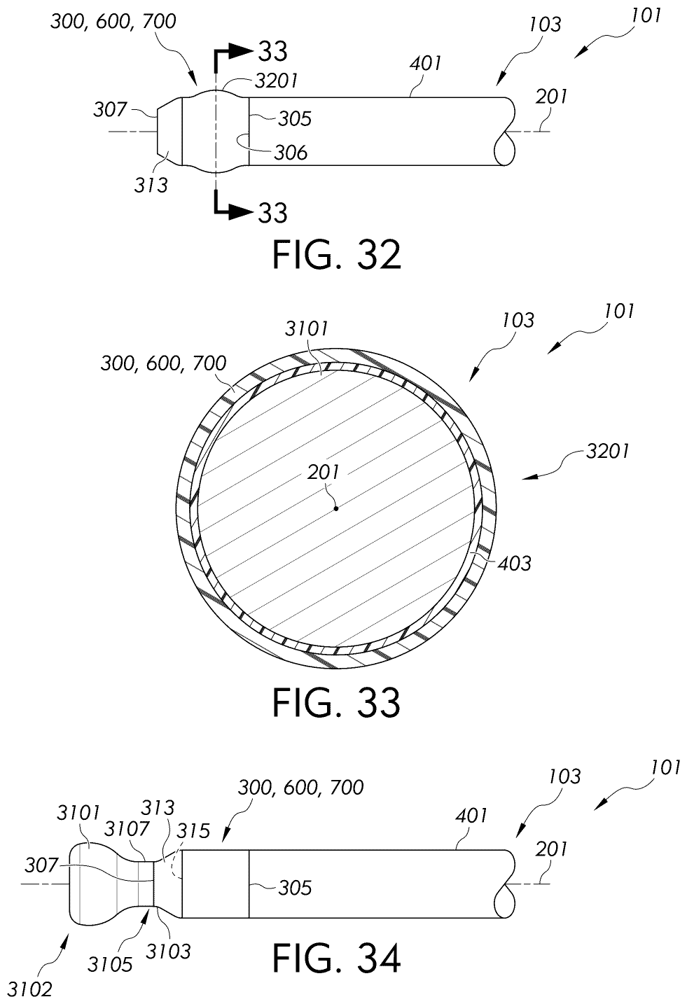

After the object 3101 has been introduced into the expandable introducer 101, during step 3509 the method can subsequently comprise distally advancing the object 3101 past the distal end 306 of the outer tubular layer 401. The radially expandable region 407 of the inner tubular layer 403 then radially expands along the axial length of the distal segment 1001 while simultaneously circumferentially stretching the flexible region 311 of the flexible tubular member 300, 600, 700. As such, once the object 3101 is advanced past the distal end 306, the distal segment 1001 of the inner tubular layer 403 and the flexible tubular member 300, 600, 700 simultaneously expand to accommodate the object 3101 passing through the inner lumen 405 of the distal segment 1001 of the inner tubular layer 403. For example, FIGS. 32-33 show the object 3101 being distally advanced past the distal end 306 of the outer tubular layer 401, wherein a portion 3201 of the expandable introducer 101 radially expands as a result of the expansion of the radially expandable region 407 and the flexible region 311.

In some aspects, the distal segment 1001 of the inner tubular layer 403 can be provided with a support member 1300, 1500, 1700. In further aspects, distally advancing the object 3101 past the distal end 306 of the outer tubular layer 401 can further comprise radially expanding the support member 1300, 1500, 1700. In some examples, the support member 1300, 1500, 1700 can radially expand simultaneously with the circumferential stretching of the flexible region 311 of the flexible tubular member 300, 600, 700. In other examples, the support member 1300, 1500, 1700 can begin radially expanding prior to the circumferential stretching of the flexible region 311, such as for example, where the support member 1300, 1500, 1700 is provided as an insert that extends at least partially into a distal end 306 of the outer tubular layer 401.

In some aspects, radially expanding the support member 1300, 1500, 1700 can further comprise radially flaring out a distal end 2802 of the support member 1300, 1500, 1700 relative to a proximal end 2804 of the support member 1300, 1500, 1700. In further aspects, the support member 1300, 1500, 1700 can comprise a self-expanding support member 1300, 1500, 1700. In some such aspects, radially flaring out the distal end 2802 can flare the support member 1300, 1500, 1700 towards its natural, expanded orientation.

During step 3511, the method can further comprise distally advancing the object 3101 past the distal end 315 of the inner tubular layer 403. For example, after the object 3101 has been distally advanced past the distal end 315 of the inner tubular layer 403, the object 3101 will begin entering the vasculature of the patient. In some examples, the object 3101 can move past the distal end 315 of the inner tubular layer 403 via a guide wire. In other examples, the object 3101 can move past the distal end 315 of the inner tubular layer 403 and into the vasculature of the patient without a guide wire.

Next, during step 3513, as shown in FIG. 34, subsequent to an enlarged end portion 3102 of the object 3101 passing through the distal end 315 of the inner tubular layer 403, the flexible tubular member 300, 600, 700 and the inner tubular layer 403 contract towards a substantially similar original orientation as they were prior to the object 3101 passing through. For example, the resiliency of the material allows for the flexible tubular member 300, 600, 700 and the inner tubular layer 403 to recontract towards their original orientation. In this way, by allowing the flexible tubular member 300, 600, 700 and the inner tubular layer 403 to recontract towards their original orientation, the expandable introducer 101 can accommodate a variety of objects featuring a range of dimensions and structural designs (e.g., a catheter-based delivery system, a dilator and/or the like). For example, the recontraction allows for subsequent entry of a smaller object 3101 following the removal of a larger object 3101 (e.g., where the larger object 3101 has a greater diameter than the smaller object 3101) while reducing or eliminating blood loss.

During step 3515, as further illustrated by FIG. 34, the method can further comprise distally advancing the object 3101 through the flexible tip 313 and past the distal end 307 of the flexible tubular member 300, 600, 700. In some aspects, the flexible tip 313 forms a rounded transition 3103 at an interface with an outer circumferential surface 3107 of the object 3101 when advancing the object 3101 past the distal end 307 as well as forming a rounded transition at the distal end 315 of the inner tubular layer 403. The rounded transitions can provide a more atraumatic feature compared to a transition with sharp edges, thereby avoiding irritation and possible vascular damage from the expandable introducer 101. In some aspects, the distal end 307 of the flexible tubular member 300, 600, 700 forms a circumferential seal 3105 against the outer circumferential surface 3107 of the object 3101 as the object 3101 passes through the flexible tip 313 and past the distal end 307 of the flexible tubular member 300, 600, 700. The circumferential seal 3105 in combination with the recontraction of the flexible tubular member 300, 600, 700 and the inner tubular layer 403 (as explained with refence to step 3513) will be especially beneficial in preventing blood loss when an object 3101 is inserted, removed, repositioned, rotated and/or otherwise adjusted. In some aspects, the flexible tip 313 can be formed in the shape of a hollow frustum in order to form the above-referenced atraumatic transitions (i.e., the rounded transition 3103, and the transition at the distal end 315 of the inner tubular layer 403).

As shown in step 3517, the method can further comprise positioning the object 3101 within a location of the patient. For example, in some aspects, the object 3101 can be a catheter delivery system, and the catheter delivery system can be inserted through the inner lumen 405 and positioned within a location of the patient's heart, for example, the location of the aortic valve (e.g., the position between the left ventricle and the aorta). Subsequently, the catheter-based delivery system can be deployed, such as for example, releasing a valve retention member from the catheter-based delivery system to deploy a prosthesis into the patient's heart.

In other embodiments, the method can further comprise proximally retracting the object 3101 within the inner lumen 405 proximally past the distal end 306 of the outer tubular layer 401 and out of the inner lumen 405 of the distal segment 1001 of the inner tubular layer 403. Referring to FIG. 34, for example, the enlarged end portion 3102 of the object 3101 can be proximally retracted relative to the flexible tubular member 300, 600, 700. The flexible tip 313 can be designed to maintain the circumferential seal 3105 against the outer circumferential surface 3107 to inhibit (e.g., prevent) blood from entering into the inner lumen 405. The flexible tip 313 can be designed to resist buckling and/or inversion when the enlarged distal end of the object 3101 reaches the distal end 307 of the flexible tubular member 300, 600, 700. Indeed, the distal tip can be fabricated from a material that has a sufficiently high axial stiffness to avoid buckling and/or inversion. Furthermore, the length “L2” can be kept sufficiently low to increase the axial stiffness as compared to longer lengths. For example, the length “L2” of the flexible tip 313 can be from about 0.5 mm to about 3 mm, for example, from about 1 mm to about 1.5 mm to avoid bucking and/or inversion when the enlarged distal end of the object 3101 is proximally retracted into the inner lumen 405 to cause the distal segment 1001 and the flexible tubular member 300, 600, 700 to radially expand to accommodate the object passing through the distal segment 1001 as shown in FIGS. 32-33. In further embodiments, as shown and described with respect to FIGS. 13-29 above, the flexible tubular member 300, 600, 700 can be provided with the support member 1300, 1500, 1700 to increase the axial stiffness to further resist buckling and/or inversion when the enlarged distal end of the object 3101 reaches the distal end 307 of the flexible tubular member 300, 600, 700.

Then, as shown in FIGS. 30-31, the object 3101 can be further proximally retracted past the distal end 306 of the outer tubular layer 401. Due to the resiliency of the flexible tubular member 300, 600, 700, the distal segment 1001 and the flexible tubular member 300, 600, 700 can then radially retract to the original nonexpanded state as shown in FIG. 30. As further shown in FIGS. 30-31, the foldable region 409 can unfold to radially expand the portion 3001 of the inner tubular layer 403 while the facing split edges of the elongated split 301 can circumferentially spread apart to further allow passage of the object 3101. In embodiments without an elongated split 301, the joining segment 402 or the entire circumference of the outer tubular layer 401 can stretch to further allow passage of the object 3101. The object 3101 can be further proximally retracted until the object is removed from the proximal end of the expandable introducer 101. Due to the resiliency of the outer tubular layer 401, once the object 3101 has passed, the facing edges of the elongated split 301 can be drawn back together (e.g., to the position shown in FIG. 4), or the joining segment 402 can resiliently retract, wherein the foldable region 409 regains the folded orientation to radially contract the inner tubular layer 403 as shown in FIG. 4. In embodiments without the elongated split, the outer tubular layer 401 can radially contract to draw back together, wherein the foldable region 409 regains the folded orientation to radially contract the inner tubular layer 403 shown in FIG. 4.

In some aspects, when proximally retracting the object 3101 (as described above) within the inner lumen 405 proximally past the distal end 306 of the outer tubular layer 401 and out of the inner lumen 405 of the distal segment 1001 of the inner tubular layer 403, the flexible tubular member 300, 600, 700 can contract to contract the radially expandable region of the distal segment 1001 of the inner tubular layer 403 as well as the support member 1300, 1500, 1700. For example, when the object 3101 is past the distal end 306 of the outer tubular layer, due to the resiliency of the flexible tubular member 300, 600, 700, the flexible tubular member 300, 600, 700 can contract, thereby constricting the support member 1300, 1500, 1700 away from its natural, expanded orientation towards a constricted orientation (e.g., as shown in FIG. 28).

In accordance with the disclosure, non-limiting aspects of the disclosure will now be described. Various combinations of the aspects can be provided in accordance with the disclosure.

Aspect 1. An expandable introducer apparatus comprising an inner tubular layer comprising an inner lumen and a radially expandable region extending along an elongated axis of the inner lumen and configured to at least partially radially expand relative to the elongated axis from a contracted orientation to an expanded orientation. An outer tubular layer comprising an inner surface bonded to the inner tubular layer, wherein a distal segment of the inner tubular layer extends beyond a distal end of the outer tubular layer. A flexible tubular member comprising a bonded region and a flexible region, wherein an inner surface of the bonded region is bonded to an outer surface of the distal segment of the inner tubular layer, the flexible region is not bonded to the radially expandable region of the inner tubular layer along an axial length of the distal segment, and the radially expandable region of the inner tubular layer along the axial length of the distal segment extends under the flexible region of the flexible tubular member.

Aspect 2. The expandable introducer apparatus of Aspect 1, wherein the flexible tubular member comprises a proximal end, a distal end, and a length extending from the proximal end to the distal end of the flexible tubular member in the direction of the elongated axis.

Aspect 3. The expandable introducer apparatus of Aspect 2, wherein the length is from about 5 mm to about 7 mm.

Aspect 4. The expandable introducer apparatus of any one of Aspects 2-3, wherein the proximal end of the flexible tubular member abuts the distal end of the outer tubular layer.

Aspect 5. The expandable introducer apparatus of any one of Aspects 2-4, wherein the flexible tubular member comprises a flexible tip that extends beyond a distal end of the inner tubular layer.

Aspect 6. The expandable introducer apparatus of Aspect 5, wherein the flexible tip comprises a length extending from the distal end of the inner tubular layer to the distal end of the flexible tubular member.

Aspect 7. The expandable introducer apparatus of Aspect 6, wherein the length of the flexible tip is from about 0.5 mm to about 3 mm.

Aspect 8. The expandable introducer apparatus of Aspect 7, wherein the length of the flexible tip is from about 1 mm to about 1.5 mm.

Aspect 9. The expandable introducer apparatus of any one of Aspects 1-8, wherein the radially expandable region of the inner tubular layer comprises a foldable region configured to be at least partially unfolded from the contracted orientation to the expanded orientation.

Aspect 10. The expandable introducer apparatus of Aspect 9, wherein the foldable region further comprises an outer fold and an inner fold in the contracted orientation.

Aspect 11. The expandable introducer apparatus of Aspect 10, wherein the flexible tubular member comprises an inner cross-sectional surface profile that matches an outer cross-sectional surface profile of the distal segment of the inner tubular layer.

Aspect 12. The expandable introducer apparatus of any one of Aspects 10-11, wherein the flexible tubular member comprises an inner circumferential flared segment comprising a flared end abutting a folded end of the outer fold of the foldable region.

Aspect 13. The expandable introducer apparatus of Aspect 10, wherein the flexible tubular member comprises a first inner protrusion aligned with a folded end of the outer fold of the foldable region and a second inner protrusion aligned with a folded end of the inner fold of the foldable region.

Aspect 14. The expandable introducer apparatus of any one of Aspects 1-10, wherein the flexible tubular member further comprises a circular cross-sectional area with a uniform wall thickness circumscribing the distal segment of the inner tubular layer.

Aspect 15. The expandable introducer apparatus of any one of Aspects 1-14, wherein the distal segment of the inner tubular layer is provided with a support member.

Aspect 16. The expandable introducer apparatus of Aspect 15, wherein the support member comprises a wire structure.

Aspect 17. The expandable introducer apparatus of Aspect 16, wherein the wire structure comprises a sinusoidal wire structure comprising a plurality of peaks circumferentially distributed around the elongated axis.

Aspect 18. The expandable introducer apparatus of Aspect 17, wherein the plurality of peaks further comprise a plurality of distal peaks and a plurality of proximal peaks.

Aspect 19. The expandable introducer apparatus of Aspect 18, wherein each peak of the plurality of distal peaks converges in a distal direction.

Aspect 20. The expandable introducer apparatus of Aspect 18, wherein each peak of the plurality of proximal peaks converges in a proximal direction.

Aspect 21. The expandable introducer apparatus of any one of Aspects 18-20, wherein a distal peak of the plurality of distal peaks comprises a first wire segment extending from the distal peak in a first direction to connect to a corresponding proximal peak of the plurality of proximal peaks, and wherein the distal peak of the plurality of distal peaks comprises a second wire segment extending from the distal peak in a second direction to connect to another corresponding proximal peak of the plurality of proximal peaks.

Aspect 22. The expandable introducer apparatus of Aspect 21, wherein the first direction comprises a first proximal direction and the second direction comprises a second proximal direction.

Aspect 23. The expandable introducer apparatus of any one of Aspects 15-22, wherein the support member extends along the axial length of the distal segment of the inner tubular layer in the direction of the elongated axis.

Aspect 24. The expandable introducer apparatus of any one of Aspects 17-23, wherein a circumferential distance between each adjacent pair of peaks of the plurality of peaks is substantially equal.

Aspect 25. The expandable introducer apparatus of any one of Aspects 15-24, wherein the support member only circumferentially extends along the bonded region of the flexible tubular member.

Aspect 26. The expandable introducer apparatus of any one of Aspects 17-24, wherein a number of peaks of the plurality of peaks in the bonded region is greater than a number of peaks of the plurality of peaks in the flexible region.

Aspect 27. The expandable introducer apparatus of any one of Aspects 15-26, wherein the support member comprises nitinol.

Aspect 28. The expandable introducer apparatus of any one of Aspects 15-27, wherein the support member is embedded within the flexible tubular member.

Aspect 29. The expandable introducer apparatus of any one of Aspects 15-28, wherein the support member is biased from radially flaring outward by a constriction of the flexible tubular member.

Aspect 30. The expandable introducer apparatus of any one of Aspects 1-29, wherein the outer tubular layer comprises an elongated split extending along the radially expandable region in a direction of the elongated axis.

Aspect 31. A method of guiding an object through an inner lumen of an expandable introducer apparatus, the expandable introducer apparatus comprising: an inner tubular layer comprising the inner lumen and a radially expandable region extending along an elongated axis of the inner lumen; an outer tubular layer comprising an inner surface bonded to the inner tubular layer, wherein a distal segment of the inner tubular layer extends beyond a distal end of the outer tubular layer; and a flexible tubular member comprising a bonded region and a flexible region, wherein an inner surface of the bonded region is bonded to an outer surface of the distal segment of the inner tubular layer, the flexible region is not bonded to the radially expandable region of the inner tubular layer along an axial length of the distal segment, and the radially expandable region of the inner tubular layer along the axial length of the distal segment extends under the flexible region of the flexible tubular member, the method comprising distally advancing the object through a proximal end of the inner lumen, wherein a portion of the radially expandable region radially expands from a contracted orientation to an expanded orientation while a corresponding portion of the outer tubular layer expands to accommodate the expanded orientation of the radially expandable region. The method further comprising distally advancing the object past the distal end of the outer tubular layer to radially expand the radially expandable region of the inner tubular layer along the axial length of the distal segment while simultaneously circumferentially stretching the flexible region of the flexible tubular member, wherein the distal segment of the inner tubular layer and the flexible tubular member simultaneously expand to accommodate the object passing through the distal segment of the inner tubular layer. Distally advancing the object past a distal end of the inner tubular layer.

Aspect 32. The method of Aspect 31, wherein a distal end of the flexible tubular member extends distally beyond the distal end of the inner tubular layer to form a flexible tip, and the method further comprises distally advancing the object through the flexible tip and past the distal end of the flexible tubular member.

Aspect 33. The method of Aspect 32, wherein the flexible tip forms a rounded transition at the distal end of the inner tubular layer when advancing the object past the distal end of the inner tubular layer and through the flexible tip.

Aspect 34. The method of any one of Aspects 32-33, wherein the distal end of the flexible tubular member forms a circumferential seal against an outer circumferential surface of the object as the object passes through the flexible tip and past the distal end of the flexible tubular member.

Aspect 35. The method of any one of Aspects 32-34, wherein a length of the flexible tip from the distal end of the inner tubular layer to the distal end of the flexible tubular member is from about 0.5 mm to about 3 mm.

Aspect 36. The method of any one of Aspects 31-35, further comprising proximally retracting the object within the inner lumen proximally past the distal end of the outer tubular layer and out of the inner lumen of the distal segment of the inner tubular layer, wherein the flexible tubular member contracts to contract the radially expandable region of the distal segment of the inner tubular layer.

Aspect 37. The method of Aspect 36, wherein the radially expandable region of the distal segment of the inner tubular layer comprises a foldable region, wherein radially contracting the flexible tubular member at least partially folds the foldable region.

Aspect 38. The method of any one of Aspects 31-36, wherein the radially expandable region comprises a foldable region, wherein radially expanding the radially expandable region at least partially unfolds the foldable region.

Aspect 39. The method of any one of Aspects 31-38, wherein the distal segment of the inner tubular layer is provided with a support member.

Aspect 40. The method of Aspect 39, wherein distally advancing the object past the distal end of the outer tubular layer further comprises radially expanding the support member.

Aspect 41. The method of Aspect 40, wherein the support member radially expands simultaneously with the circumferential stretching of the flexible region of the flexible tubular member.

Aspect 42. The method of any one of Aspects 40-41, wherein radially expanding the support member further comprises radially flaring out a distal end of the support member relative to a proximal end of the support member.

Aspect 43. The method of Aspect 42, wherein the support member comprises a self-expanding support member and radially flaring out the distal end flares the support member towards its natural, expanded orientation.

Aspect 44. The method of any one of Aspects 40-43, further comprising proximally retracting the object within the inner lumen proximally past the distal end of the outer tubular layer and out of the inner lumen of the distal segment of the inner tubular layer, wherein the flexible tubular member contracts to contract the radially expandable region of the distal segment of the inner tubular layer and the support member.

Aspect 45. The method of any one of Aspects 31-44, wherein the outer tubular layer comprises an elongated split extending along the radially expandable region in a direction of the elongated axis, and the distally advancing the object through the proximal end of the inner lumen radially expands the portion of the radially expandable region while a corresponding portion of the elongated split separates.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. An expandable introducer apparatus comprising:
an inner tubular layer comprising an inner lumen and a radially expandable region extending along an elongated axis of the inner lumen and configured to at least partially radially expand relative to the elongated axis from a contracted orientation to an expanded orientation;
an outer tubular layer comprising an inner surface bonded to the inner tubular layer, wherein a distal segment of the inner tubular layer extends beyond a distal end of the outer tubular layer; and
a flexible tubular member comprising a bonded region and a flexible region, wherein an inner surface of the bonded region is bonded to an outer surface of the distal segment of the inner tubular layer, the flexible region is not bonded to the radially expandable region of the inner tubular layer along an axial length of the distal segment, and the radially expandable region of the inner tubular layer along the axial length of the distal segment extends under the flexible region of the flexible tubular member.

2. The expandable introducer apparatus of claim 1, wherein a proximal end of the flexible tubular member abuts a distal end of the outer tubular layer.

3. The expandable introducer apparatus of claim 1, wherein the flexible tubular member comprises a flexible tip that extends beyond a distal end of the inner tubular layer.

4. The expandable introducer apparatus of claim 3, wherein a length of the flexible tip is from about 0.5 mm to about 3 mm.

5. The expandable introducer apparatus of claim 1, wherein the radially expandable region of the inner tubular layer comprises a foldable region configured to be at least partially unfold from the contracted orientation to the expanded orientation.

6. The expandable introducer apparatus of claim 5, wherein the foldable region further comprises an outer fold and an inner fold in the contracted orientation.

7. The expandable introducer apparatus of claim 1, wherein the distal segment of the inner tubular layer is provided with a support member.

8. The expandable introducer apparatus of claim 7, wherein the support member comprises a wire structure.

9. The expandable introducer apparatus of claim 8, wherein the wire structure comprises a sinusoidal wire structure comprising a plurality of peaks circumferentially distributed around the elongated axis.

10. A method of guiding an object through an inner lumen of an expandable introducer apparatus, the expandable introducer apparatus comprising: an inner tubular layer comprising the inner lumen and a radially expandable region extending along an elongated axis of the inner lumen; an outer tubular layer comprising an inner surface bonded to the inner tubular layer, wherein a distal segment of the inner tubular layer extends beyond a distal end of the outer tubular layer; and a flexible tubular member comprising a bonded region and a flexible region, wherein an inner surface of the bonded region is bonded to an outer surface of the distal segment of the inner tubular layer, the flexible region is not bonded to the radially expandable region of the inner tubular layer along an axial length of the distal segment, and the radially expandable region of the inner tubular layer along the axial length of the distal segment extends under the flexible region of the flexible tubular member, the method comprising:
distally advancing the object through a proximal end of the inner lumen, wherein a portion of the radially expandable region radially expands from a contracted orientation to an expanded orientation while a corresponding portion of the outer tubular layer expands to accommodate the expanded orientation of the radially expandable region;
distally advancing the object past the distal end of the outer tubular layer to radially expand the radially expandable region of the inner tubular layer along the axial length of the distal segment while simultaneously circumferentially stretching the flexible region of the flexible tubular member, wherein the distal segment of the inner tubular layer and the flexible tubular member simultaneously expand to accommodate the object passing through the distal segment of the inner tubular layer; and
distally advancing the object past a distal end of the inner tubular layer.

11. The method of claim 10, wherein a distal end of the flexible tubular member extends distally beyond the distal end of the inner tubular layer to form a flexible tip, and the method further comprises distally advancing the object through the flexible tip and past the distal end of the flexible tubular member.

12. The method of claim 11, wherein the flexible tip forms a rounded transition at the distal end of the inner tubular layer when advancing the object past the distal end of the inner tubular layer and through the flexible tip.

13. The method of claim 11, wherein the distal end of the flexible tubular member forms a circumferential seal against an outer circumferential surface of the object as the object passes through the flexible tip and past the distal end of the flexible tubular member.

14. The method of claim 11, wherein a length of the flexible tip from the distal end of the inner tubular layer to the distal end of the flexible tubular member is from about 0.5 mm to about 3 mm.

15. The method of claim 10, wherein the radially expandable region comprises a foldable region, wherein radially expanding the radially expandable region at least partially unfolds the foldable region.

16. The method of claim 10, wherein the distal segment of the inner tubular layer is provided with a support member.

17. The method of claim 16, wherein distally advancing the object past the distal end of the outer tubular layer further comprises radially expanding the support member.

18. The method of claim 17, wherein the support member radially expands simultaneously with the circumferential stretching of the flexible region of the flexible tubular member.

19. The method of claim 17, wherein radially expanding the support member further comprises radially flaring out a distal end of the support member relative to a proximal end of the support member.

20. The method of claim 19, wherein the support member comprises a self-expanding support member and radially flaring out the distal end flares the support member towards its natural, expanded orientation.
